(19) United States
(12) Patent Application Publication
(10) Pub. No.: US 2026/0315400 A1
(43) Pub. Date: Oct. 15, 2026

(54) PROCESS FOR PRODUCING MICROPARTICLES

(71) Applicant: BASF SE, Ludwigshafen am Rhein (DE)

(72) Inventors: Pierre-Eric MILLARD, Ludwigshafen (DE); Zhibing ZHANG, Birmingham (GB); Siddhant Pravin BHUTKAR, Ludwigshafen (DE); Jon PREECE, Birmingham (GB)

(21) Appl. No.: 19/104,161

(22) PCT Filed: Aug. 15, 2023
(86) PCT No.: PCT/EP2023/072457

§ 371 Date: Feb. 14, 2025

(30) Foreign Application Priority Data
Aug. 18, 2022 (EP) 22191053.2

(51) Int. Cl. A61K 9/50; A61K 8/11; A61Q 13/00; C11D 17/00

(52) U.S. Cl. CPC A61K 9/5089; A61K 8/11; A61Q 13/00; C11D 17/0039; A61K 2800/56; A61K 2800/614

(57) ABSTRACT

The present invention relates to a process for producing microparticles comprising at least one organic active compound encapsulated by a shell of an organic wall material, which comprises
i. providing a water-immiscible liquid containing the organic active compound to be encapsulated and at least a first shell forming compound (SFC1);
ii. emulsifying the water-immiscible liquid obtained in step i. in an aqueous medium to obtain an oil-in-water (o/w) emulsion of the water-immiscible liquid in the aqueous medium;
iii. adding at least one second shell forming compound (SFC2) to the aqueous medium before or during carrying out step ii. or to the emulsion obtained in step ii., whereby an aqueous suspension of the microparticles is obtained;
where either the first shell forming compound (SFC1) or the second shell forming compound (SFC2) has 1, 2, 3, 4, 5 or 6, in particular 1, 2, 3 or 4, especially 1, 2 or 3 first reactive groups (RG1) per molecule, which are selected from isocyanate groups, isothiocyanate groups, carbonyl halide groups and carboxylic anhydride groups, while the other shell forming compound has 1, 2, 3, 4, 5 or 6, in particular 1, 2, 3 or 4, especially 1, 2 or 3 second reactive groups (RG2) per molecule which are selected from hydroxyl groups, thiol groups and primary amino groups, where either the first shell forming compound (SFC1) or the second shell forming compound (SFC2) has only one reactive group while the other shell forming compound has 1, 2, 3, 4, 5 or 6, in particular 1, 2, 3 or 4, especially 1, 2 or 3 reactive groups.

The present invention also relates to the microparticles obtainable by this process and to their uses.

The present invention relates to a process for producing microparticles comprising at least one organic active compound encapsulated by a shell of an organic wall material formed by an in situ-encapsulation process using a combination of two different reactive shell forming compounds, where the reactive functional groups of the different shell forming compounds have a complementary reactivity such that they react with each other by forming covalent bonds. Also claimed are microparticles comprising at least one organic active compound encapsulated by a shell of an organic wall material, where the wall material is a urea compound or an amide compound obtained by combination of two different reactive shell forming material.

Microcapsules have various uses as carriers for active substances like crop protecting agents, pharmaceutical agents, fragrances, aromas, and also for reactive substances or catalysts for industrial applications. They typically comprise a polymeric material that envelops the material to be encapsulated. Advantages of a formulation of this kind are in particular:
protection of reactive actives from environmental effects;
safe and practical handling of toxic or unstable actives;
controlled release of actives;
prevention of premature mixing of substances;
the handling of liquid actives as solids and reciprocally.

An overview of the methodology of microencapsulation of actives can be found in H. Mollet, A. Grubenmann, “Formulation Technology”, chapter 6.4 (Microencapsulation), pages 234-246, Wiley VCH Verlag GmbH, Weinheim 2001, and the literature cited therein.

In many agricultural and cosmetic products, it is desirable for aroma chemicals and agrochemical actives to be released slowly over time. For example, in case of perfume materials, the more volatile perfume raw materials are responsible for the “fresh feeling” that consumers experience. In case of agrochemicals, it is often desired to achieve a long lasting effect, to reduce volatility and/or to reduce negative environmental effects or acute toxicity effects by controlled release of the respective active. Therefore, it is desirable for these materials to be released in a slow, controlled manner.

One frequently used approach for the controlled release of the respective active is the microencapsulation. Typically, these microcapsules comprise a hydrophobic liquid core containing the respective active which is surrounded by a synthetic polymer shell. Such polymers may be, for example, a polyurethane, polyurea, polyamide, polyester, polycarbonate, urea/formaldehyde resin, a melamine/formaldehyde resin, a polystyrene, or an acrylate polymer. The microparticles are typically prepared by an interfacial polymerization of the respective monomers or oligomers, which form the polymer shell in an oil-in-water (o/w) emulsion of the respective hydrophobic liquid.

Methods for encapsulating organic liquids containing aroma chemicals by urea/formaldehyde resins or melamine formaldehyde resins have been described e. g. in WO 2008/066773 and WO 2009/090169 and the references cited therein.

Methods for encapsulating organic liquids containing agrochemicals by in situ formation of polyurea or polyurethanes have been described e. g. in U.S. Pat. Nos. 5,705,174, 5,910,314, WO 94/13139, WO 2015/165834 and WO 2018/130588.

The synthetic polymers of the microcapsules remain in the environment as microplastics and are therefore not ecologically harmless (see e. g. C. M. Rochmann, “The global odyssee of plastic pollution, Science 368 (2020) pp 1184-1185). The use of synthetic polymer microcapsules is therefore increasingly meeting with ecological concerns on the part of customers and the regulatory authorities. Therefore, there is a demand for providing delivery forms for organic actives which can be produced without non-degradable plastic material or with reduced amounts of non-degradable plastic material.

WO 2018/065481 and WO 2019/193094 disclose a process for preparing microparticles laden with organic compounds, such as aroma chemicals. The process comprises producing porous microparticles made of a thermoplastic biodegradable polyester material and suspending the microparticles in a liquid aroma chemical or a solution of the aroma chemical. The porous microparticles are produced by providing an w/o-emulsion of the polyester material dissolved in a water-immiscible solvent as a continuous phase and an aqueous solution of a pore-forming agent as the discontinuous phase, emulsifying the w/o-emulsion in water to obtain a water-in-oil-in-water (w/o/w) emulsion and removing the organic solvent by evaporation. In order to achieve controlled release, the microparticles laden with the aroma chemical must be closed or sealed, which is effected by heating the laden microparticles over a time-prolonged period, which can lead to degradation of the active and unwanted agglomeration or even destruction of the laden microparticles. Moreover, the release characteristics are not always satisfactory. Moreover, several steps have to be carried out.

The aforementioned production methods for microparticles laden with an aroma chemical require still or contain polymer material which may ultimately incompletely degrade or degrade over a very long time period and thus may contribute to microplastics in the environment. Apart from that their production is tedious and requires either prolonged heating of the laden microparticles which may be detrimental to the microparticles and/or the aroma chemical or the presence of a coating or matrix. Furthermore, the production usually requires large amounts of chlorinated hydrocarbon solvents. Also, the preparation of such particles is carried out in separate steps, one being the preparation of hollow microparticles, followed by loading with desired substances and prolonged heating to prevent premature exit of the substance. Thus, alternative processes are required to simplify the production process.

Self-assembly of organic diamide diacids in aqueous phase to form hollow microparticles has been reported by R. J. Bergeron et al, Bioorganic & Medicinal chemistry, Vol. 5, 11 (1997), pp. 2049-2061 and O. Phanstiel et al., Chem Mater. 2001, 13, 264-272. Self-assembly is achieved by lowering the pH of an aqueous alkaline solution of the diacids. This process results in suspensions of microparticles, which enclose the aqueous phase. Thus, it is not suitable for encapsulating organic actives, in particular organic actives which are sparingly or insoluble in water.

S. R. Wilson-Withfordd et al, ACS Appl. Mater. Interfaces 2021, 13, 4, 5887-5894 describe the preparation of microcapsules through crystallization of diurethane compounds, such as decane-1,10-bis(cyclohexyl carbamate) (DBCC). For this, DBCC is prepared by reacting cyclohexylisocyanate with 1,10-decanediol in dry chloroform in the presence of a tin catalyst, whereby DBCC is formed within three hours. DBCC is then dissolved in a 9:1 mixture of dichloromethane/decane at a concentration of 0.1% by weight. The solution is then injected into an aqueous phase and after evaporation of dichloromethane, an aqueous suspension of microcapsules is formed which contains decane within a capsule shell formed by crystallized DBCC. The method is tedious and requires large amounts of chlorinated hydrocarbons, which must be evaporated.

It is therefore an object of the invention to provide a simple and straightforward process for preparing microparticles laden with at least one organic chemical which does not require the presence of a polymer matrix or coating. The microparticles are to have a relatively low surface porosity causing slow-release properties. The laden microparticles are to be producible in a simple process and with high yield but without significant agglomeration or even destruction of the laden microparticles.

It has been found that, surprisingly, these and further objects are achieved by the process described hereinafter and the organic chemical-filled microparticles that are obtainable thereby.

The present invention therefore relates to a process for producing microparticles comprising at least one organic active compound encapsulated by a shell of an organic wall material, which comprises
i. providing a water-immiscible liquid containing the organic active compound to be encapsulated and at least a first shell forming compound (SFC1);
ii. emulsifying the water-immiscible liquid obtained in step i. in an aqueous medium to obtain an oil-in-water (o/w) emulsion of the water-immiscible liquid in the aqueous medium;
iii. adding at least one second shell forming compound (SFC2) to the aqueous medium before or during carrying out step ii. or to the emulsion obtained in step ii., whereby an aqueous suspension of the microparticles is obtained;
where either the first shell forming compound (SFC1) or the second shell forming compound (SFC2) has 1, 2, 3, 4, 5 or 6, in particular 1, 2, 3 or 4, especially 1, 2 or 3 first reactive groups (RG1) per molecule, which are selected from isocyanate groups, isothiocyanate groups, carbonyl halide groups and carboxylic anhydride groups, while the other shell forming compound has 1, 2, 3, 4, 5 or 6, in particular 1, 2, 3 or 4, especially 1, 2 or 3 second reactive groups (RG2) per molecule which are selected from hydroxyl groups, thiol groups and primary amino groups, where either the first shell forming compound (SFC1) or the second shell forming compound (SFC2) has only one reactive group while the other shell forming compound has 1, 2, 3, 4, 5 or 6, in particular 1, 2, 3 or 4, especially 1, 2 or 3 reactive groups.

Thereby, microparticles are obtained where the shell of the organic wall material is formed by the reaction of the at least one first shell forming compound (SFC1) with the at least one second shell forming compound (SFC2). In other words, the wall material of the microparticles obtainable by the process according to the invention essentially consist of the reaction product of the at least one first shell forming compound (SFC1) with the at least one second shell forming compound (SFC2). In this context, the term “essentially” is clearly understood that the portion of the organic wall material which is formed by the reaction of the first shell forming compound with the second shell forming compound amounts to a least 90% by weight, based on the total weight of the organic matter in the wall material.

The microparticles which are obtainable by this process are novel and have particularly beneficial properties, when the wall material is formed by urea compounds or amide compounds or thiourethane compounds or thiourea compounds. Such a wall material is formed in step iii), if the functional groups RG2 are primary amino groups or thiol groups. Particular preference is given to microparticles which are obtainable by this process, if the wall material is formed by urea compounds, which are obtainable, if the functional groups (RG1) are isocyanate groups and the functional groups (RG2) are primary amino groups.

Therefore, the present invention also relates to microparticles comprising at least one organic compound encapsulated by a shell of an organic wall material formed by the reaction of a first shell forming compound (SFC1) with a second shell forming compound (SFC2),
where either the first shell forming compound (SFC1) or the second shell forming compound (SFC2) has 1, 2, 3, 4, 5 or 6, in particular 1, 2, 3 or 4, especially 1, 2 or 3 first reactive groups (RG1) per molecule, which are selected from isocyanate groups, isothiocyanate groups, carbonyl halide groups and carboxylic anhydride groups, while the other shell forming compound has 1, 2, 3, 4, 5 or 6, in particular 1, 2, 3 or 4, especially 1, 2 or 3 second reactive groups (RG2) per molecule which are selected from primary amino groups and thiol groups,
where either the first shell forming compound (SFC1) or the second shell forming compound (SFC2) has only one reactive group while the other shell forming compound has 1, 2, 3, 4, 5 or 6, in particular 1, 2, 3 or 4, especially 1, 2 or 3 reactive groups.

The present invention in particular relates to microparticles comprising at least one organic compound encapsulated by a shell of an organic wall material formed by the reaction of at least one first shell forming compound (SFC1) with at least one second shell forming compound (SFC2), which are obtainable by the process of the present invention,
where either the first shell forming compound (SFC1) or the second shell forming compound (SFC2) has 1, 2, 3, 4, 5 or 6, in particular 1, 2, 3 or 4, especially 1, 2 or 3 first reactive groups (RG1) per molecule, which are selected from isocyanate groups, isothiocyanate groups, carbonyl halide groups and carboxylic anhydride groups, while the other shell forming compound has 1, 2, 3, 4, 5 or 6, in particular 1, 2, 3 or 4, especially 1, 2 or 3 second reactive groups (RG2) per molecule which are primary amino groups or thiol groups,
where either the first shell forming compound (SFC1) or the second shell forming compound (SFC2) has only one reactive group while the other shell forming compound has 2, 3, 4, 5 or 6, in particular 2, 3 or 4, especially 2 or 3 reactive groups.

The invention is associated with a number of advantages:
The process of the present invention produces microparticles laden with an organic active compound which allow for controlled release of the organic active compound, namely by burst release caused by mechanical pressure, by diffusion or by degradation of the biodegradable membrane which leads to slow release effects desirable for certain applications.
The wall material of the microparticles obtainable by the process is a nonpolymeric material of low and defined molecular weight because at least one of the shell forming material has only one reactive functionality and thus no polymeric material is formed in the process.
The process of the present invention is much simpler, more straightforward and less problematic than comparable processes requiring separate steps of producing hollow particles, followed by filling the microparticles with an aroma chemical and finally sealing the porous surface of the pores e.g. by applying heat or a coating.
In particular, the process of the invention does not require volatile organic solvents, in particular no halogenated hydrocarbon solvent for the encapsulation of the organic active compound. In particular, no evaporation step is required to induce the shell formation via self-assembly. Therefore, the process is easier to carry out and does not cause health risk, does not require additional solvent recycling and limits waste management.
By the process of the invention, the surface porosity of the microparticles laden with the organic active compound is greatly reduced without significantly destroying or agglomerating the laden microparticles due to extended exposure to heat.
As the surface porosity of the laden microparticles is reduced, they can be stored over a prolonged period without any significant loss of the organic active compound, which is in particular important in case of sensitive actives and/or volatile organic actives.

The present invention also relates to use of the microparticles as described herein which contain an encapsulated aroma chemical as an additive for imparting a scent or a flavour to a product which is e. g. selected from perfumes, washing and cleaning products, cosmetic products, personal care products, hygiene articles, foods, food supplements, fragrance dispensers and fragrances.

The present invention also relates to the use of the microparticles as described herein for controlled release of the organic active compound. In particular, the present invention relates to use of the microparticles as described herein which contain an encapsulated aroma chemical or an agrochemical for controlled release of aroma chemicals or agrochemicals, respectively.

Here and throughout this application the terms “organic active compound”, “organic active” and “active compound” are used synonymously. The terms are understood by the person skilled in the art to mean an organic chemical compound that triggers a physiological effect in living beings and plants, and substances that cause a chemical effect or catalyze a chemical reaction in inanimate nature. Examples of actives are aroma chemicals, organic crop protecting agents, organic pharmaceutical agents, organic cosmetic actives and organic actives for uses in the construction sector, called construction chemicals, especially catalysts for products in the construction sector, e.g. crosslinking or polymerization catalysts. The term “organic active” also includes “metal organic actives”.

The term “volatile organic active” refers to an organic or organometallic chemical compound having a boiling temperature or evaporation temperature of at most 250° C. at 101.3 kPa and/or a vapor pressure at 20° C. of at least 50 Pa.

Here and in the following, the term “water-immiscible” refers to a material, whose solubility in deionized water at 20° C. and 1 bar is at most 5 g/L, in particular at most 1 g/L. The solubility of the water-immiscible material in deionized water under the conditions given here may be zero, i. e. below the detection limit.

The term liquid refers to a material in the liquid state at ambient conditions, i. e. to a non-solid and non-gaseous material. In the context of the invention, a liquid material preferably has a dynamic viscosity at 20° C. in the range of 0.2 to 2000 mPas, in particular in the range of 0.5 to 1000 mPas. Here and throughout the specification, ambient conditions refer to a temperature in the range of 20-25° C. and atmospheric pressure, i. e. about 1 bar.

The term “organic active compound of low molecular weight” refers to an organic or organometallic chemical active compound having a defined molecular weight Mn which is generally below 1000 daltons and typically in the range from 80 to <1000 daltons and especially in the range from 100 to 500 daltons. The molecular weight can be determined by mass spectroscopy.

The term “sensitive actives” refers to organic active compounds which are not stable to the conditions of their environment and are damaged or degrade, for example due to pH of their environment or by oxidation.

In the context of the shell forming compounds (SFC1) and (SFC2) a skilled person will clearly understand the term “isocyanate group” to mean a functional group of the formula —N═C═O. The term “isothiocyanate group” refers to a functional group of the formula —N═C═S. The term “carbonyl halide group” refers to a group of the formula —C(═O)—X, where X refers to halogen, in particular to chlorine or bromine, which may also refer to as carboxylic acid halide group. The term carboxylic anhydride group refers to a group of the formula —C(═O)—O—C(═O)—R, where R refers to hydrogen or a hydrocarbon radical having preferably from 1 to 6 carbon atoms, such as C₁-C₆ alkyl or phenyl. The term “primary amino group” is clearly understood to mean a functional group of the formula —NH₂. The term “hydroxyl group” is clearly understood to mean a functional group of the formula —OH. The term “thiol group” is clearly understood to mean a functional group of the formula —SH. The aforementioned functional groups are bound to carbon atoms of the shell forming compounds (SFC1) and (SFC2), respectively.

Unless stated otherwise, the term “room temperature” indicates a temperature of 22° C.

According to the invention, shell forming compounds (SFC1) have either reactive groups (RG1), i. e. groups selected from isocyanate groups, isothiocyanate groups, carbonyl halide groups carboxyl anhydride groups, or reactive groups (RG2), i. e. hydroxyl groups and/or primary amino groups and/or thiol groups, with RG2 being preferably primary amino groups or a combination of primary amino groups and hydroxyl groups, with particular preference being primary amino groups.

Consequently, the shell forming compounds (SFC2) must have 1, 2, 3, 4, 5 or 6, in particular 1, 2, 3 or 4, especially 1, 2 or 3 reactive groups (RG2) per molecule, if the shell forming compound (SFC1) has 1, 2, 3, 4, 5 or 6, in particular 1, 2, 3 or 4, especially 1, 2 or 3 reactive groups (RG1) per molecule, provided that either the shell forming compound (SFC1) has only one reactive group (RG1) per molecule or the shell forming compound (SFC2) has only one reactive group (RG2) per molecule, while the other shell forming compound has 1, 2, 3, 4, 5 or 6 reactive groups, in particular 1, 2, 3 or 4, especially 1, 2 or 3 complementary reactive groups per molecule. Likewise, the shell forming compounds (SFC2) must have 1, 2, 3, 4, 5 or 6, in particular 1, 2, 3 or 4, especially 1, 2 or 3 reactive groups (RG1) per molecule, if the shell forming compound (SFC1) has 1, 2, 3, 4, 5 or 6, in particular 1, 2, 3 or 4, especially 1, 2 or 3 reactive groups (RG2) per molecule, provided that either the shell forming compound (SFC1) has only one reactive group (RG2) or the shell forming compound (SFC2) has only one reactive group (RG1) per molecule, while the other shell forming compound has 1, 2, 3, 4, 5 or 6 reactive groups, in particular 1, 2, 3 or 4, especially 1, 2 or 3 complementary reactive groups per molecule.

The reactive groups present in the shell forming compounds (SFC2) thus have a complementary reactivity to the reactive group present in the shell forming compound (SFC1). Complementary reactivity means that the reactive groups (RG1) and (RG2) are capable of reacting with each other by forming a covalent bond between the reactive centers of the reactive groups (RG1) and (RG2). As a result, in step iii) the first shell forming compound (SFC1) contained in the emulsified water-immiscible liquid reacts with second shell forming compound (SFC2) present in the aqueous phase of the o/w emulsion at the surface of the droplets of the o/w emulsion. Thereby a wall material is formed at the surface of the droplets of the water-immiscible liquid which forms the shell of the microcapsules and thus encapsulates the droplets of the water-immiscible liquid which contains the organic active compound.

It is immediately clear that the process of the invention requires that either
(a) the first shell forming compound (SFC1) contained in the water-immiscible liquid containing the organic active compound to be encapsulated has exactly 1 reactive group (RG1) or (RG2) per molecule if the second shell forming compound (SCF2) added in step iii) has 1, 2, 3, 4, 5 or 6 reactive groups having a reactivity which is complementary to the reactivity of the reactive groups of the first shell forming compound (SFC1)—hereinafter group (a) of embodiments;
or
(b) the second shell forming compound (SFC2) added in step iii has exactly 1 reactive group (RG1) or (RG2) per molecule if the first shell forming compound (SCF1) contained in the water-immiscible liquid containing the organic active compound to be encapsulated has 1, 2, 3, 4, 5 or 6 reactive groups having a a reactivity which is complementary to the reactivity of the reactive groups of the second shell forming compound (SFC2)—hereinafter group (b) of embodiments.

It is also apparent that in the group (a) of embodiments the water-immiscible liquid containing the organic active compound to be encapsulated is essentially free of shell forming compounds having more than 1 reactive group (RG1) or (RG2) per molecule while in the group (b) of embodiments, essentially no shell forming compound is added which has more than 1 reactive group (RG1) or (RG2) per molecule. In particular, in group (a) of embodiments, the water-immiscible liquid containing the organic active compound to be encapsulated does not contain more than 10% by weight, in particular not more than 5% by weight of shell forming compounds having more than 1 reactive group (RG1) or (RG2) per molecule, based on the total amount of shell forming compounds contained in the water-immiscible liquid. In other words, in groups (a) of embodiments, the relative amount of the shell forming compound (SCF1) having exactly 1 reactive group (RG1) or (RG2) is at least 90% by weight, in particular at least 95% by weight, based on the total amount of shell forming compounds contained in the water-immiscible liquid. Likewise, in group (b) of embodiments, the relative amount of shell forming compound (SFC2) having exactly one reactive group (RG1) or (RG2) is at least 90% by weight, in particular at least 95% by weight or 100% by weight, based on the total amount of shell forming compounds added in step iii. of the process of the invention.

Accordingly, in group (b) of embodiments the relative amount of shell forming compounds having more than one reactive group (RG1) or (RG2) is at most 10% by weight, in particular at most 5% by weight, based on the total amount of shell forming compounds added in step iii.

It is also immediately clear that the process of the invention will also work, if the shell forming compound (SFC1) is a mixture of different shell forming compounds (SFC1) having the same reactivity and/or if the shell forming compound (SFC2) is a mixture of different shell forming compounds (SFC2) having the same reactivity. It is also clear from the foregoing, that the shell forming compounds (SFC1) and (SFC2) must not have reactive groups other than reactive groups (RG1) and (RG2).

Depending on the chosen combination of complementary reactive groups of the shell forming compounds (SFC1) and (SFC2), the organic wall material or shell material is
a urea compound having 1, 2, 3, 4, 5 or 6, in particular 2, 3, or 4 urea groups, especially 2 or 3 urea groups, i. e. —NH—C(═O)—NH— groups,
an urethane compound having 1, 2, 3, 4, 5 or 6, in particular 2, 3, or 4 urethane groups, especially 2 or 3 urethane groups, i. e. —NH—C(═O)—O— groups,
a thiourethane compound having 1, 2, 3, 4, 5 or 6, in particular 2, 3, or 4 thiourethane groups, especially 2 or 3 thiourethane groups, i. e. —NH—C(═O)—S— groups,
a thiourea compound having 1, 2, 3, 4, 5 or 6, in particular 2, 3, or 4 thiourea groups, especially 2 or 3 thiourea groups, i. e. —NH—C(═S)—NH— groups,
an amide compound having 1, 2, 3, 4, 5 or 6, in particular 2, 3, or 4 carboxamide groups, especially 2 or 3 carboxamide groups, i. e. —C(═O)—NH— groups.

With respect to the stability of the shell, the complementary reactive groups of the shell forming compounds (SFC1) and (SFC2) are preferably chosen such that the organic wall material or shell material is formed by urea compounds, thiourea compounds, urethane compounds, thiourethane compounds or carboxamide compounds. In particular, the complementary reactive groups of the shell forming compounds (SFC1) and (SFC2) are chosen such that the organic wall material or shell material is formed by urea compounds, thiourea compounds, urethane compounds, thiourethane compounds or carboxamide compounds having 2, 3 or 4, preferably 2 or 3, especially 2 groups, selected from urea groups, thiourea groups, urethane groups, thiourethane groups and carboxamide groups.

More preferably, the complementary reactive groups of the shell forming compounds (SFC1) and (SFC2) are chosen such that the organic wall material or shell material is formed by urea compounds or urethane compounds having 1, 2, 3, 4, 5 or 6, in particular 2, 3 or 4, preferably 2 or 3, especially 2 groups, selected from urea groups and urethane groups. Most preferably, the complementary reactive groups of the shell forming compounds (SFC1) and (SFC2) are chosen such that the organic wall material or shell material is formed by urea compounds, in particular by urea compounds having 2, 3 or 4, preferably 2 or 3, especially 2 urea groups.

In these cases, the process is preferably carried out such that the first shell forming compound (SFC1) has one reactive group (RG1) which is an isocyanate group, while the second shell forming compound (SFC2) has 1, 2, 3, 4, 5 or 6, in particular 2, 3 or 4, preferably 2 or 3 and especially 2 primary amino groups or hydroxyl groups, whereby microparticles are obtained, wherein the wall material or shell material, respectively, is formed by compounds having 1, 2, 3, 4, 5 or 6, in particular 2, 3, or 4 urea groups or urethane groups, preferably 2 or 3 urea or urethane groups and especially 2 urea or urethane groups.

For producing microparticles, where the organic wall material is formed by compounds having 1, 2, 3, 4, 5 or 6, in particular 2, 3, or 4 carboxamide groups, preferably 2 or 3 carboxamide, especially 2 carboxamide groups the process is preferably carried out, such the first shell forming compound (SFC1) has one reactive group (RG1) which is a carbonyl halide group or a carboxylic anhydride group, in particular a carbonyl chloride group, while the second shell forming compound (SFC2) has 1, 2, 3, 4, 5 or 6, in particular 2, 3 or 4, preferably 2 or 3, especially 2 primary amino groups, whereby microparticles are obtained, wherein the organic wall material or shell material, respectively, is formed by compounds having 2, 3, or 4 carboxamide groups, especially 2 or 3 carboxamide groups.

In another preferred group of embodiments, the complementary reactive groups of the shell forming compounds (SFC1) and (SFC2) are chosen such that the organic wall material or shell material, respectively, is formed by thiourea compounds, in particular by thiourea compounds having 1, 2, 3, 4, 5 or 6, in particular 2, 3 or 4, preferably 2 or 3, especially 2 thiourea groups, respectively. In these cases, the process is preferably carried out such that the first shell forming compound (SFC1) has one reactive group (RG1) which is an isothiocyanate group, while the second shell forming compound (SFC2) has 1, 2, 3, 4, 5 or 6, in particular 2, 3 or 4, preferably 2 or 3 and especially 2 primary amino groups, whereby microparticles are obtained, wherein the organic wall material or shell material, respectively, is formed by compounds having 1, 2, 3, 4, 5 or 6, in particular 2, 3, or 4 thiourea groups, preferably 2 or 3 thiourea groups and especially 2 thiourea groups.

In yet a further preferred group of embodiments, the complementary reactive groups of the shell forming compounds (SFC1) and (SFC2) are chosen such that the organic wall material or shell material, respectively, is formed by thiourethane compounds, in particular by thiourethane compounds having 1, 2, 3, 4, 5 or 6, in particular 2, 3 or 4, preferably 2 or 3, especially 2 thiourethane groups, respectively.

In these cases, the process is preferably carried out such that the first shell forming compound (SFC1) has one reactive group (RG1) which is an isocyanate group, while the second shell forming compound (SFC2) has 1, 2, 3, 4, 5 or 6, in particular 2, 3 or 4, preferably 2 or 3 and especially 2 thiol groups, whereby microparticles are obtained, wherein the organic wall material or shell material, respectively, is formed by compounds having 1, 2, 3, 4, 5 or 6, in particular 2, 3, or 4 thiourethane groups, preferably 2 or 3 thiourethane groups and especially 2 thiourethane groups.

Especially, the complementary reactive groups of the shell forming compounds (SFC1) and (SFC2) are chosen such that the organic wall material or shell material is formed by urea compounds having 2 urea groups. In this case, the process is preferably carried out by using shell forming compound (SFC1) having one reactive group (RG1) which is an isocyanate group, while the second shell forming compound (SFC2) having 2 reactive groups (RG2) which are primary amino groups.

The shell forming compounds (SFC1) and (SFC2) are generally low molecular weight compounds, which typically have a molecular weight of not more than 1000 g/mol, e. g. in the range of 60 to 500 g/mol, in particular in the range of 80 to 400 g/mol. Since the number of reactive sites of the respective shell forming compound (SFC1) and (SFC2) is limited to a maximum of 6 and the at least one of the shell forming compounds (SFC1) and (SFC2), respectively has only one reactive group, the resulting wall material is formed by compounds having a maximum molecular weight of 2000 g/mol and typically at most 1600 g/mol with particular preference given to maximum molecular weights in the range of 200 to 1600 g/mol, especially in the range of 200 to 1200 g/mol. Here, the term “maximum” molecular weight refers to the molecular weight that is not exceeded by 90% by weight of the molecules forming the organic wall material. The maximum molecular weight of the organic wall material can be determined by well established methods of high-performance liquid chromatography coupled to mass spectrometry (HPLC-MS).

For the efficiency of the encapsulation process, it is beneficial, if the first shell forming compound has 1, 2, 3, 4, 5 or 6, in particular 1, 2, 3 or 4, preferably 1, 2 or 3, more preferably 1 or 2, especially 1 first reactive groups (RG1). In this case, the second shell forming compound (SFC2) has 1, 2, 3, 4, 5 or 6, in particular 1, 2, 3 or 4, preferably 1, 2 or 3, more preferably 1 or 2, especially 2 second reactive groups (RG2) per molecule, provided that either the first shell forming compound (SFC1) or the second shell forming compound (SFC2) has only one reactive group while the other shell forming compound has 1, 2, 3, 4, 5 or 6, in particular 1, 2, 3 or 4, preferably 1, 2 or 3, more preferably 2 or 3, especially 2 complementary reactive groups. In particular the reactive groups (RG1) are isocyanate groups.

For the efficiency of the encapsulation process, it is particularly beneficial, if the first shell forming compound (SFC1) has 1 first reactive group (RG1) per molecule, while the second shell forming compound (SFC2) has 1, 2, 3, 4, 5 or 6, in particular 1, 2, 3 or 4, more particularly 2 or 3, especially 2 second reactive groups (RG2) per molecule. In this case, the first reactive group (RG1) is in particular an isocyanate group.

It is also beneficial, if the first shell forming compound (SFC1) has 1, 2, 3, 4, 5 or 6, in particular 1, 2, 3 or 4, more particularly 2 or 3, especially 2 first reactive groups (RG1) per molecule, while the second shell forming compound (SFC2) has 1 second reactive group (RG2) per molecule. In this case, the first reactive group (RG1) is in particular an isocyanate group.

Preferably, the combination of the shell forming compounds (SFC1) and (SFC2) is chosen in a manner such that urethane, urea or amide groups are formed by the reaction of the shell forming compounds (SFC1) with the shell forming compounds (SFC2).

Therefore, a particular group (1) of embodiments relates to the process of the invention where the first reactive groups (RG1) are isocyanate groups and the second reactive groups (RG2) are hydroxyl groups or in particular a primary amino groups, while another group (2) of embodiments relate to the process of the invention where the first reactive groups (RG1) are carbonyl halide or carboxyl anhydride groups and the second reactive groups (RG2) are primary amino groups.

A further particular group (3) of embodiments relates to the process of the invention where the first reactive groups (RG1) are isothiocyanate groups and the second reactive groups (RG2) are hydroxyl groups or in particular a primary amino groups.

Suitable combinations are given in the following table 1, where RG indicates the type of reactive groups present in the shell forming compounds (SFC) and (SFC2), respectively, and n indicates the numbers of reactive groups (RG) present in the shell forming compounds (SFC2) and (SFC2), respectively. A skilled person will immediately understand that lines 1 to 8 of table 1 relate to group (1) of embodiments, while the combinations given in lines 9 to 12 of table 1 relate to group (2) of embodiments and the combinations given in lines 13 to 18 of table 1 relate to group (3) of embodiments.

TABLE 1

| | SFC1 | | SFC2 | |
|---|---|---|---|---|
| # | RG | n | RG | n |
| 1 | N═C═O | 1 | NH₂ | 1, 2, 3 or 4, in particular |
| | | | | 2 or 3, especially 2 |
| 2 | N═C═O | 1 | OH | 1, 2, 3 or 4, in particular |
| | | | | 2 or 3, especially 2 |
| 3 | N═C═O | 1, 2, 3 or 4, in | NH₂ | 1 |
| | | particular 2 or 3, | | |
| | | especially 2 | | |
| 4 | N═C═O | 1, 2, 3 or 4, in | OH | 1 |
| | | particular 2 or 3, | | |
| | | especially 2 | | |
| 5 | NH₂ | 1, 2, 3 or 4, in | N═C═O | 1 |
| | | particular 2 or 3, | | |
| | | especially 2 | | |
| 6 | OH | 1, 2, 3 or 4, in | N═C═O | 1 |
| | | particular 2 or 3, | | |
| | | especially 2 | | |
| 7 | NH₂ | 1 | N═C═O | 1, 2, 3 or 4, in particular |
| | | | | 2 or 3, especially 2 |
| 8 | OH | 1 | N═C═O | 1, 2, 3 or 4, in particular |
| | | | | 2 or 3, especially 2 |
| 9 | C(O)—X | 1 | NH₂ | 1, 2, 3 or 4, in particular |
| | with | | | 2 or 3, especially 2 |
| | X═Cl, Br | | | |
| 10 | C(O)—X | 1, 2, 3 or 4, in | NH₂ | 1 |
| | with | particular 2 or 3, | | |
| | X═Cl, Br | especially 2 | | |
| 11 | NH₂ | 1, 2, 3 or 4, in | C(O)—Cl | 1 |
| | | particular 2 or 3, | | |
| | | especially 2 | | |
| 12 | NH₂ | 1 | C(O)—Cl | 1, 2, 3 or 4, in particular |
| | | | | 2 or 3, especially 2 |
| 13 | N═C═O | 1 | SH | 1, 2, 3 or 4, in particular |
| | | | | 2 or 3, especially 2 |
| 14 | N═C═O | 1, 2, 3 or 4, in | SH | 1 |
| | | particular 2 or 3, | | |
| | | especially 2 | | |
| 15 | N═C═S | 1 | NH₂ | 1, 2, 3 or 4, in particular |
| | | | | 2 or 3, especially 2 |
| 16 | N═C═S | 1 | OH | 1, 2, 3 or 4, in particular |
| | | | | 2 or 3, especially 2 |
| 17 | N═C═S | 1, 2, 3 or 4, in | NH₂ | 1 |
| | | particular 2 or 3, | | |
| | | especially 2 | | |
| 18 | N═C═S | 1, 2, 3 or 4, in | OH | 1 |
| | | particular 2 or 3, | | |
| | | especially 2 | | |

Amongst the combinations given in table 1, particular preference is given to the combinations given in lines 1 to 4 and in lines 9 to 10 with more preference given to the combinations of lines 1 and 3.

Suitable shell forming compounds having reactive groups (RG1) include but are not limited to
monoisocyanates of the group of aliphatic monoisocyanates, cycloaliphatic monoisocyanates, aromatic monoisocyanates and araliphatic monoisocyanates;
diisocyanates of the group of aliphatic diisocyanates, cycloaliphatic diisocyanates, aromatic diisocyanates and araliphatic diisocyanates;
triisocyanates of the group of aliphatic triisocyanates, cycloaliphatic triisocyanates, aromatic triisocyanates and araliphatic triisocyanates;
monoacyl chlorides of the group of aliphatic monoacylchlorides, cycloaliphatic monoacyl chlorides, aromatic monoacylchlorides and araliphatic monosacylchlorides;
diacyl chlorides of the group of aliphatic diacylchlorides, cycloaliphatic bisacyl chlorides, aromatic bisacylchlorides and araliphatic bisacylchlorides;
monoisothiocyanates of the group of aliphatic monoisothiocyanates, cycloaliphatic monoisothiocyanates, aromatic monoisothiocyanates and araliphatic monoisothiocyanates;
diisothiocyanates of the group of aliphatic diisothiocyanates, cycloaliphatic diisothiocyanates, aromatic diisothiocyanates and araliphatic diisothiocyanates; and
triisothiocyanates of the group of aliphatic triisothiocyanates, cycloaliphatic triisothiocyanates, aromatic triisothiocyanates and araliphatic triisothiocyanates.

In the aforementioned shell forming compounds having reactive groups (RG1) the isocyanate groups, isothiocyanate groups and the carbonyl chloride groups are usually bound to a carbon atom of an aliphatic, cycloaliphatic, aromatic or araliphatic radical having from 2 to 20 carbon atoms, wherein 1, 2 or 3 non-adjacent CH₂ groups in the aliphatic and cycloaliphatic radicals may be replaced by oxygen atoms or N-methyl groups and where in the aliphatic, cycloaliphatic, aromatic or araliphatic radical 1, 2 or 3 non-adjacent CH groups may be replaced by N. Such radicals include C₁-C₂₀ alkyl radicals, C₅-C₂₀ cycloalkyl radicals, which are optionally substituted by 1, 2, 3 or 4 C₁-C₄ alkyl radicals, aryl radicals, such as benzene radicals, which are optionally substituted by 1, 2, 3 or 4 C₁-C₄ alkyl radicals, phenylalkylbenzene radicals which are optionally substituted by 1, 2, 3 or 4 C₁-C₄ alkyl radicals and diphenylether radicals which are optionally substituted by 1, 2, 3 or 4 C₁-C₄ alkyl radicals.

Here and throughout the specification, the prefixes Cₙ-Cₘ used in connection with compounds or molecular moieties each indicate a range for the number of possible carbon atoms that a molecular moiety or a compound can have. The term “C₁-Cₙ alkyl” denominates a group of linear or branched saturated hydrocarbon radicals having from 1 to n carbon atoms. The term “Cₙ/Cₘ alkyl” denominates a mixture of two alkyl groups, one having n carbon atoms while the other having m carbon atoms.

For example, the term C₁-C₂₀ alkyl denominates a group of linear or branched saturated hydrocarbon radicals having from 1 to 20 carbon atoms, while the term C₁-C₄ alkyl denominates a group of linear or branched saturated hydrocarbon radicals having from 1 to 4 carbon atoms and the C₅-C₂₀ alkyl denominates a group of linear or branched saturated hydrocarbon radicals having from 5 to 20 carbon atoms. Examples of alkyl include but are not limited to methyl, ethyl, n-propyl, isopropyl, n-butyl, sec-butyl, isobutyl, tert-butyl, 2-methylpropyl (isopropyl), 1,1-dimethylethyl (tert-butyl), pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 2-heptyl, n-octyl, 2-octyl, 2-ethylhexyl, nonyl, isononyl, decyl, undecyl, dodecyl, tridecyl, isotridecyl, tetradecyl, pentadecyl, hexadecyl, heptadecyl, octadecyl, nonadecyl, eicosyl, heneicosyl docosyl and in case of nonyl, isononyl, decyl, undecyl, dodecyl, tridecyl, isotridecyl, tetradecyl, pentadecyl, hexadecyl, heptadecyl, octadecyl, nonadecyl, eicosyl, heneicosyl docosyl their isomers, in particular mixtures of isomers such as “isononyl”, “isodecyl”. Examples of C₁-C₄-alkyl are for example methyl, ethyl, propyl, 1-methylethyl, butyl, 1-methylpropyl, 2-methylpropyl or 1,1-dimethylethyl.

The term “C₅-C₂₀-cycloalkyl” as used herein refers to a saturated mono- or polycyclic, in particular mono-, bi- or tricyclic (cycloaliphatic) radical which is unsubstituted or substituted by 1, 2, 3 or 4 C₁-C₄ alkyl radicals, where the total number of carbon atoms of C₅-C₂₀-cycloalkyl from 5 to 20 and where the total number of ring-forming atoms is preferably in the range of 3 to 16. Examples of C₅-C₂₀-cycloalkyl include but are not limited to cyclopentyl, cyclohexyl, methylcyclohexyl, dimethylcyclohexyl, cycloheptyl, cyclooctyl, cyclododecyl, cyclohexadecyl, norbornyl (=bicyclo[2.2.1]heptyl) and isobornyl (=1,7,7-trimethylbicyclo[2.2.1]heptyl). In “C₅-C₂₀-cycloalkyl” 1, 2 or 3 non-adjacent ring-forming CH₂ groups may be replaced by oxygen atoms while the remainder of the ring-forming atoms are carbon atoms. These groups are also referred to as C₃-C₂₀-heterocycloalkyl and where the total number of carbon atoms in C₃-C₂₀-heterocycloalkyl is in the range of 3 to 20. Examples of such radicals include, but are not limited to oxolan-2-yl, oxolan-3-yl, oxan-2-yl, oxan-3-yl, oxan-4-yl, 1,3-dioxolan-2-yl, 1,3-dioxolan-4-yl, 2-methyl-1,3-dioxolan-4-yl, 2,2-dimethyl-1,3-dioxolan-4-yl, 1,4-dioxan-2-yl, 1,3-dioxan-2-yl, 1,3-dioxan-4-yl, 1,3-dioxan-5-yl, 2-methyl-1,3-dioxan-4-yl, 2-methyl-1,3-dioxan-5-yl, 2,2-dimethyl-1,3-dioxan-4-yl, 2,2-dimethyl-1,3-dioxan-5-yl, 2,3,3a,5,6,6a-hexahydrofuro[3,2-b]furan-2-yl, 2,3,3a,5,6,6a-hexahydrofuro[3,2-b]furan-3-yl and 2,5-dioxabicyclo[2,2,1]heptan-7-yl.

Aryl refers to phenyl, napthyl and diphenyl, which are optionally substituted by 1, 2, 3 or 4 C₁-C₄ alkyl radicals, Araliphatic radicals, also denominated as arylalkyl, refer to phenyl and naphthyl, optionally substituted by 1, 2, 3 or 4 C₁-C₄ alkyl radicals which bear a further alkylene group. Examples of arylalkyl include benzyl, phenethyl, phenylalkylbenzene radicals and diphenylether radicals where the phenyl rings in the 4 mentioned groups are optionally substituted by 1, 2, 3 or 4 C₁-C₄ alkyl radicals.

Examples of suitable monoisocyanates include C₁-C₂₀ alkyl isocyanates, such as methyl isocyanate, ethyl isocyanate, butyl isocyanate, pentyl isocyanate, hexyl isocyanate, heptyl isocyanate, octyl isocyanate, nonyl isocyanate, deccyl isocyanate, C₅-C₂₀ cycloalkyl isocyanates, such as cyclohexyl isocyanate, cycloheptyl isocyanate, and aryl isocyanates such as phenyl isocyanate and tolyl isocyanate.

Examples of suitable diisocyanates include C₂-C₂₀ alkylene diisocyanates, such as hexamethylene diisocyanate, tetramethylene diisocyanate, 1,8-diisocyanatooctate, 1,10-diisocyanato decane, C₂-C₂₀ cycloalkylene diisocyanates, such as isophorone diisocyanate, dicyclohexylmethane-4,4′-diisocyanate, aryl diisocyanates, such as 2,4- and 2,6-toluylene diisocyanate and isomer mixtures thereof, and arylalkyl diisocyanates, such as 2,4′- and 4,4′-diphenylmethane diisocyanate and mixtures thereof.

Examples of suitable triisocyanates include the biureths and the isocyanurates of the aforementioned diisocyanates.

Examples of suitable monoacyl chlorides include C₁-C₂₀ alkanoyl chlorides, such as acetyl chloride, propionyl chloryl, butyryl chloride, pentanoic acid chloride, hexanoic acid chloride, heptanoic acid chloride, octanoic acid chloride, nonanoic acid chloride, decanoic acid chloride, C₅-C₂₀ cycloalkanoyl chlorides, such as cyclohexanoic acid chloride and aromatic carboxylic acid chlorides, such as benzoyl chloride.

Examples of suitable diacyl chlorides include succinic acid dichloride, glutaric acid dichloride, adipic acid dichloride, pimelic acid dichloride, suberic acid dichloride, azelaic acid dichloride, sebacic acid dichloride, brassylic acid dichloride, cyclohex-andioic acid dichloride, phthalic acid dichloride and isophthalic acid dichloride.

Suitable shell forming compounds having reactive groups (RG2) include but are not limited to
primary monoamines selected from the group of aliphatic monoamines, cycloaliphatic monoamines and aromatic monoamines,
primary diamino compounds selected from the group of aliphatic diamines, cycloaliphatic diamines and aromatic diamines;
primary triamino compounds selected from the group of aliphatic triamines, cycloaliphatic triamines, and aromatic triamines;
primary tetraamino compounds selected from the group of aliphatic tetramines, cycloaliphatic tetramines, and aromatic tetramines;
monoalcohols selected from the group of aliphatic monoalcohols, cycloaliphatic monoalcohols and aromatic monoalcohols;
dialcohol selected from the group of aliphatic diols, cycloaliphatic diols, aromatic diols and araliphatic diols;
trialcohols selected from the group of aliphatic triols, cycloaliphatic triols, aromatic triols and araliphatic triols;
tetraalcohols selected from the group of aliphatic tetraols, cycloaliphatic tetraols, aromatic tetraols and araliphatic tetraols
monothiols selected from the group of aliphatic monothiols, cycloaliphatic monothiols and aromatic monothiols;
dithiols selected from the group of aliphatic dithiols, cycloaliphatic dithiols, aromatic dithiols and araliphatic dithiols;
trithiols selected from the group of aliphatic trithiols, cycloaliphatic trithiols, aromatic trithiols and araliphatic trithiols; and
tetrathiols selected from the group of aliphatic tetrathiols, cycloaliphatic tetrathiols, aromatic tetrathiols and araliphatic tetrathiols.

In the aforementioned shell forming compounds having reactive groups (RG2) primary amino groups, the thiol groups and the hydroxyl groups are usually bound to a carbon atom of an aliphatic, cycloaliphatic or aromatic radical having from 2 to 20 carbon atoms, wherein 1, 2 or 3 non-adjacent CH₂ groups in the aliphatic and cycloaliphatic radicals may be replaced by oxygen atoms and where in the aliphatic, cycloaliphatic or aromatic radicals 1, 2 or 3 non-adjacent CH groups may be replaced by N. Such radicals include C₁-C₂₀ alkyl radicals, C₅-C₂₀ cycloalkyl radicals, which are optionally substituted by 1, 2, 3 or 4 C₁-C₄ alkyl radicals, C₆-C₂₀ bicycloalkyl radicals, which are optionally substituted by 1, 2, 3 or 4 C₁-C₄ alkyl radicals, aryl radicals, such as benzene radicals, which are optionally substituted by 1, 2, 3 or 4 C₁-C₄ alkyl radicals, phenylalkylbenzene radicals which are optionally substituted by 1, 2, 3 or 4 C₁-C₄ alkyl radicals and diphenylether radicals which are optionally substituted by 1, 2, 3 or 4 C₁-C₄ alkyl radicals and 1,3,5-triazine radicals.

Examples of suitable monoamines include C₁-C₂₀ alkyl amine, such as methyl amine, ethyl amine, butyl amine, pentyl amine, hexyl amine, heptyl amine, octyl amine, nonyl amine, deccyl amine, C₅-C₂₀ cycloalkyl amine, such as cyclohexyl amine, cycloheptyl amine, and aryl amines such as phenyl amine, benzylamine, phenethylamine and tolyl amine.

Examples of suitable diamines include diamino C₂-C₂₀ alkanes, such as diamino ethane, 1,3-diamino propane, 1,4-diamino butane, 1,5-diamino pentane, 1,6-diamino hexane, 1,8-diamino octane, 1,10-diamino decane, 1,12-diaminododecane, N-methyl-N-(3-aminopropyl)-1,3-diaminopropane, 3-oxa-1,5-diaminopentane, 3,6-dioxa-1,8-diaminooctane, 4,7-dioxa-1,10-diaminooctane, diamino-C₅-C₂₀ cycloalkanes, such as 1,2-, 1,3- and 1,4-diaminocyclohexanes and isophorone diamine, and aryl amines such as o-, m- and p-diaminobenzene and the diaminotoluene isomers.

Examples of suitable triamines include N,N-bis(3-aminopropyl)-1,3-diaminopropane, N,N-bis(2-aminoethyl)-1,3-diaminopropane and 2,4,6-triamino-s-triazine.

Examples of suitable tetraamines include N,N,N′,N′-tetrakis(3-aminopropyl)-1,3-diaminopropane, N,N,N′,N′-tetrakis-(2-aminoethyl)-1,3-diaminopropane and tetraaminobenzene.

Examples of suitable monoalcohols include C₁-C₂₀ alkanols, such as methanol, ethanol, n-propanol, iso-propanol, n-butanol, 2-butanol, isobutanol, tert-butanol, n-pentanol, isoamyl alcohol, n-hexanol, 2-hexanol, n-heptanol, n-octanol, isooctanol, 2-ethylhexan-1-ol, n-nonanol, isononanol, ethyleneglycol-monomethylether, ethyleneglycol-monoethylether, 1,3-propyleneglycol-monomethylether, 1,3-propyleneglycol-monoethylether, cyclopentanol, cyclohexanol, cycloheptanol, cyclooctanol, cyclodecanol, phenol and benzyl alcohol.

Examples of suitable dialcohol include C₂-C₂₀ alkanediols, such as 1,2-ethylene glycol, 1,2-propanediol, 1,3-propanediol, 1,4-butanediol, 1,5-pentanediol, 1,6-hexanediol, diethylene glycohol, triethyleneglycol, tripropyleneglycol, cycloaliphatic diols, such as 1,2-, 1,3- and 1,4-cyclohexanediol, 1,2-, 1,3- and 1,4-cyclohexanedimethanol, aromatic diols, such as resorcin, and araliphatic diols, such as p-hydroxybenzyl alcohol and 1,2-, 1,3- and 1,4-bis(hydroxymethyl)benzene.

Examples of suitable trialcohols include C₃-C₂₀ alkanetriols, such as glycerine, 2,2-bis(hydroxymethyl)-1-ethanol, 2,2-bis(hydroxymethyl)-1-propanol, 2,2-bis(hydroxymethyl)-1-butanol, triethanol amine tripropanolamine.

Examples of suitable tetraalcohols include C₃-C₂₀ alkanetraols, such as pentaerythrite, N,N,N′,N′-tetrakis-(2-hydroxyethyl)-1,2-diaminoethane, N,N,N′,N′-tetrakis-(2-hydroxyethyl)-1,3-diaminopropane, N,N,N′,N′-tetrakis-(2-hydroxyethyl)-1,4-diaminobutane, N,N,N′,N′-tetrakis-(2-hydroxyethyl)-1,5-diaminopentane, N,N,N′,N′-tetrakis-(2-hydroxyethyl)-1,6-diaminohexane, N,N,N′,N′-tetrakis-(2-hydroxypropyl)-1,2-diaminoethane, N,N,N′,N′-tetrakis-(2-hydroxypropyl)-1,3-diaminopropane, N,N,N′,N′-tetrakis-(2-hydroxypropyl)-1,4-diaminobutane, N,N,N′,N′-tetrakis-(2-hydroxypropyl)-1,5-diaminopentane and N,N,N′,N′-tetrakis-(2-hydroxypropyl)-1,6-diaminohexane.

Examples of suitable monothiols include C₁-C₂₀ alkanethiols, such as thiomethanol, thioethanol, n-propyl mercaptan, iso-propyl mercaptan, n-butyl mercaptan, 2-thiobutanol, isobutyl mercaptan, tert-butanyl mercaptan, n-pentyl mercaptan, isopentyl mercaptan, n-hexyl mercaptan, 2-hexylmercaptan, n-heptyl mercaptan, n-octyl mercaptan, isooctylmercaptan and thiophenol.

Examples of suitable dithiols include C₂-C₂₀ alkanedithiols, such as 1,2-ethane dithiol, 1,2-propane dithiol, 1,3-propane dithiol, 1,4-butane dithiol, 1,5-pentane dithiol, 1,6-hexane dithiol, cycloaliphatic dithiols, such as 1,2-, 1,3- and 1,4-cyclohexane dithiol, aromatic dithiols, such as benzene dithiols and biphenyl-4,4-dithiol.

In a preferred group (a.1) of embodiments, the first shell forming compound (SFC1) is selected from the group consisting of monoisocyanates, i. e. compounds having exactly 1 isocyanate group (NCO) per molecule, including aliphatic monoisocyanates, cycloaliphatic monoisocyanates and aromatic monoisocyanates and where the second shell forming compound (SFC2) is selected from the group consisting of aliphatic diamines, aliphatic triamines, aliphatic tetramines, aliphatic pentamines, aliphatic hexamines, cycloaliphatic diamines, cycloaliphatic triamines, cycloaliphatic tetramines, cycloaliphatic pentamines, cycloaliphatic hexamines, aromatic diamines, aromatic triamines, aromatic tetramines, aromatic pentamines and aromatic hexamines. In this very preferred group of embodiments, the second shell forming compound (SFC2) is in particular selected from the group consisting of aliphatic diamines, aliphatic triamines, cycloaliphatic diamines, cycloaliphatic triamines, aromatic diamines and aromatic triamines, with particular preference given to aliphatic diamines, aliphatic triamines, and aromatic triamines, such as melamine (2,4,6-triamino-s-triazine).

In a particularly preferred group (a.11) of embodiments, the first shell forming compound (SFC1) is selected from the group consisting of aliphatic monoisocyanates, cycloaliphatic monoisocyanates and aromatic monoisocyanates and where the second shell forming compound (SFC2) is selected from the group consisting of aliphatic diamines. In this very preferred group of embodiments, the second shell forming compound (SFC2) is in particular selected from the group consisting of diamino C₄-C₁₆ alkanes, such as 1,4-diamino butane, 1,5-diamino pentane, 1,6-diamino hexane, 1,8-diamino octane, 1,10-diamino decane and 1,12-diaminododecane.

In a further preferred group (b.1) of embodiments, the first shell forming compound (SFC1) is selected from the group consisting of isocyanates having at least 2 NCO groups per molecule, in particular 2, 3 or 4 a NCO groups per molecule and where the second shell forming compound (SFC2) is selected from the group consisting of aliphatic monoamines, cycloaliphatic monoamines and aromatic monoamines. In this preferred group group (b.1) of embodiments, the isocyanates having at least 2 NCO may be in particular aliphatic, aromatic or cycloaliphatic diisocynates, in particular C₂-C₂₀ alkylene diisocyanates, such as hexamethylene diisocyanate, tetramethylene diisocyanate, 1,8-diisocyanatooctate, 1,10-diisocyanato decane, C₂-C₂₀ cycloalkylene diisocyanates, such as isophorone diisocyanate, dicyclohexylmethane-4,4′-diisocyanate, aryl diisocyanates, such as 2,4- and 2,6-toluylene diisocyanate and isomer mixtures thereof, and arylalkyl diisocyanates, such as 2,4′- and 4,4′-diphenylmethane diisocyanate and mixtures thereof.

The relative amount of the shell forming materials (SFC1) and (SFC2) is preferably chosen such that the molar ratio of the reactive groups (RG1) to (RG2) present in the shell forming materials (SFC1) and (SFC2) is close to the required stoichiometry. The reactive groups (RG1), may however be used in a certain excess or deficit. Preferably, the molar ratio of the total amount of reactive groups (RG1) to the total amount of reactive groups (RG2) is in the range of 1.0:2.0 to 2.0:1.0, in particular in the range of 1.0:1.6 to 1.6:1.0, especially in the range of 1.0:1.3 to 1.3:1.0.

The total amount of the shell forming compounds (SFC1) and (SFC2) is preferably chosen such that the weight ratio of the total amount of the shell forming compounds (SFC1) and (SFC2) to the water-immiscible liquid is in the range of 1:1 to 1:50, in particular in the range of 1:1 to 1:25 and especially in the range of 1:1 to 1:10. Accordingly, the total amount of shell material in the final microcapsules is frequently in the range of 2 to 50% by weight, in particular in the range of 4 to 40% by weight and especially in the range of 5 to 40% by weight, based on the total weight of the final microcapsules, i. e. based on the total weight of the water-immiscible liquid and the shell material resulting from the reaction of the shell forming compound (SFC1) with the shell forming compound (SFC2).

In the process of the present invention, the organic active compound to be encapsulated and the first shell forming compound (SFC1) are present in the water-immiscible liquid. Here and in the following, the term “water-immiscible liquid” refers to a liquid, whose solubility in deionized water at 20° C. and 1 bar is at most 5 g/L, in particular at most 1 g/L. The miscibility of the water-immiscible liquid with deionized water under the conditions given here may be zero, i. e. below the detection limit.

The organic active compound to be encapsulated and the first shell forming compound (SFC1) may form the water-immiscible liquid, i. e. the organic active compound to be encapsulated and the first shell forming compound (SFC1) constitute more than 99% by weight of the total weight of the water-immiscible liquid. Alternatively, the organic active compound may be present dissolved or dispersed in a water-immiscible organic solvent, in particular if the organic active compound is not a liquid at 25° C. and 1 bar. Preferably, the organic active compound to be encapsulated and the first shell forming compound (SFC1) are mutually dissolved in each other or they are a solution in a water-immiscible organic solvent.

The term “water-immiscible organic solvent” refers to an organic solvent, whose solubility in deionized water at 20° C. and 1 bar is at most 5 g/L, in particular at most 1 g/L. The miscibility of the water-immiscible organic solvent with deionized water under the conditions given here may be 0, i. e. below the detection limit.

In particular, the water immiscible liquid is a solution of the organic compound to be encapsulated and the first shell forming compound (SFC1) and optionally one or more organic water-immiscible solvents.

Suitable water-immiscible organic solvent are in particular hydrocarbons, such as aliphatic and cycloaliphatic organic solvents and aromatic solvents and mixtures thereof, esters of C₂-C₂₀-mono- or dicarboxylic acids with C₁-C₁₀ alkanols having a total of at least 6 carbon atoms, such as butyl acetate, hexyl acetate, iso nonyl acetate, isopropyl myristate, C₆-C₁₀ alkanols, fatty acid amides having a total of at least 8 carbon atoms, such as dimethylhexanamide and dimethyloctanamide, and mixtures of the foregoing organic solvents.

Preferably, the organic solvent has a boiling point of at least 100° C., e. g. in the range of 100 to 350° C. at 1 bar.

The organic active compound may principally be any active compound, which is either liquid at 20° C. and 1 bar or which is capable to be dissolved or dispersed in a water-immiscible organic solvent. In particular, the organic active is non-polar and has a high affinity to the water-immiscible liquid. In particular, the organic active has an octanol-water partition coefficient K_{ow}=Cₒ/C_{w} of greater than 0, in particular of at least 1.5 at 20° C.

In a particular group of embodiments, the organic active compound is liquid at 22° C. and 1 bar or a mixture of two or more organic active compounds which is liquid at 22° C. and 1 bar.

In another particular group of embodiments, the organic active is solid at 22° C. and 1 bar or a mixture of two or more organic active compounds which is solid at 22° C. and 1 bar. In this case, the organic active compound or the mixture of the two or more organic active compounds is provided as a solution in a water immiscible compound.

The organic active compound may be a single compound or a mixture of compounds, e. g. a mixture of 2, 3 or more active compounds.

The organic active compound is frequently a low molecular weight organic active compound or a mixture of low molecular weight organic active compounds.

The organic active compound typically does not have a reactive group which is reactive towards the reactive groups (RG1) and (RG2).

The organic active compound may be selected from agrochemicals, aromachemicals, pharmaceutically active compounds, vitamins, cosmetic actives and and organic effect compounds, such as polymerization catalysts, dyes and UV filters.

In a preferred group of embodiments, the organic active is an aroma chemical, especially an aroma chemical which is liquid at 22° C. and 1 bar or a mixture of two or more aroma chemicals which is liquid at 22° C. and 1 bar. Preferred aroma chemicals are hydrophobic and, especially at 25° C., have a water solubility in deionized water of not more than 100 mg/L. In another preferred group of embodiments, the organic active is an aroma chemical which is solid at 22° C. and 1 bar or a mixture of two or aroma chemicals which is solid at 22° C. and 1 bar. In this case, the aroma chemical or the mixture of the two or more aroma chemicals is provided as a solution in a water immiscible compound.

The term “aroma chemical” is understood by the person skilled in the art to mean organic compounds usable as “odorant” and/or as “flavoring”. In the context of the present invention, “odorant” is understood to mean natural or synthetic substances having intrinsic odor. In the context of the present invention, “flavoring” is understood to mean natural or synthetic substances having intrinsic flavor. In the context of the present invention, “odor” or “olfactory perception” is the interpretation of the sensory stimuli which are sent from the chemoreceptors in the nose or other olfactory organs to the brain of a living being. The odor can be a result of sensory perception of the nose of odorant, which occurs during inhalation. In this case, the air serves as odor carrier.

Preferred aroma chemicals for loading of the microparticles are selected, for example, from the following compounds:
alpha-hexylcinnamaldehyde, 2-phenoxyethyl isobutyrate (Phenirat¹), dihydromyrcenol (2,6-dimethyl-7-octen-2-ol), methyl dihydrojasmonate (preferably having a cis isomer content of more than 60% by weight) (Hedione⁹, Hedione HC⁹), 4,6,6,7,8,8-hexamethyl-1,3,4,6,7,8-hexahydrocyclopenta[g]benzopyran (Galaxolide³), tetrahydrolinalool (3,7-dimethyloctan-3-ol), ethyl linalool, benzyl salicylate, 2-methyl-3-(4-tert-butylphenyl)propanal (Lilial²), cinnamyl alcohol, 4,7-methano-3a,4,5,6,7,7a-hexahydro-5-indenyl acetate and/or 4,7-methano-3a,4,5,6,7,7a-hexahydro-6-indenyl acetate (Herbaflorat¹), citronellol, citronellyl acetate, tetrahydrogeraniol, vanillin, linalyl acetate, styrenyl acetate (1-phenylethyl acetate), octahydro-2,3,8,8-tetramethyl-2-acetonaphthone and/or 2-acetyl-1,2,3,4,6,7,8-octahydro-2,3,8,8-tetramethylnaphthalene (Iso E Super₁), hexyl salicylate, 4-tert-butylcyclohexyl acetate (Oryclone¹), 2-tert-butylcyclohexyl acetate (Agrumex HC¹), alpha-ionone (4-(2,2,6-trimethyl-2-cyclohexen-1-yl)-3-buten-2-one), n-alpha-methylionone, alpha-isomethylionone, coumarin, terpinyl acetate, 2-phenylethyl alcohol, 4-(4-hydroxy-4-methylpentyl)-3-cyclohexenecarboxaldehyde (Lyral³), alpha-amylcinnamaldehyde, ethylene brassylate, (E)- and/or
(Z)-3-methylcyclopentadec-5-enone (Muscenone⁹), 15-pentadec-11-enolide and/or
15-pentadec-12-enolide (Globalide¹), 15-cyclopentadecanolide (Macrolide¹), 1-(5,6,7,8-tetrahydro-3,5,5,6,8,8-hexamethyl-2-naphthalenyl)ethanone (Tonalide¹°), 2-isobutyl-4-methyltetrahydro-2H-pyran-4-ol (Florol⁹), 2-ethyl-4-(2,2,3-trimethyl-3-cyclopenten-1-yl)-2-buten-1-ol (Sandolene¹), cis-3-hexenyl acetate, trans-3-hexenyl acetate, trans-2-cis-6-nonadienol, 2,4-dimethyl-3-cyclohexenecarboxaldehyde (Vertocitral¹), 2,4,4,7-tetramethyloct-6-en-3-one (Claritone¹), 2,6-dimethyl-5-hepten-1-al (Melonal²), borneol, 3-(3-isopropylphenyl)butanal (Florhydral²), 2-methyl-3-(3,4-methylenedioxyphenyl)propanal (Helional³), 3-(4-ethylphenyl)-2,2-dimethylpropanal (Florazon¹), tetrahydro-2-isobutyl-4-methyl-2H-pyran (Dihydrorosenon⁴),
1,4-bis(ethoxymethyl)cyclohexane (Vertofruct⁴), L-isopulegol (1R,2S,5R)-2-isopropenyl-5-methylcyclohexanol, pyranyl acetate (2-isobutyl-4-methyltetrahydropyran-4-yl acetate), nerol ((Z)-2,6-dimethyl-2,6-octadien-8-ol), neryl acetate, 7-methyl-2H-1,5-benzodioxepin-3(4H)-one (Calone¹⁹⁵¹⁵), 3,3,5-trimethylcyclohexyl acetate (preferably with a content of cis isomers of 70% by weight) or more and 2,5,5-trimethyl-1,2,3,4,4a,5,6,7-octahydronaphthalen-2-ol (Ambrinol S¹), tetrahydro-4-methyl-2-(2-methylpropenyl)-2H-pyran (rose oxide), 4-methyl-2-(2-methylpropyl)oxane or 4-methyl-2-(2-methylpropyl)-2H-pyran (Dihydrorosan⁴), prenyl acetate (=3-methylbut-2-enyl acetate), isoamyl acetate, dihydromyrcenol (2,6-dimethyloct-7-en-2-ol) and methylheptenone (6-methylhept-5-en-2-one) and mixtures thereof, and also mixtures thereof with one or more other aromas.

In the context of the present invention, the aforementioned aromas or odorants are accordingly preferably combined with mixtures of the invention.

If trade names are specified above, these refer to the following sources:
¹ trade name of Symrise GmbH, Germany;
² trade name of Givaudan AG, Switzerland;
³ trade name of International Flavors & Fragrances Inc., USA;
⁴ trade name of BASF SE;
⁵ trade name of Danisco Seillans S.A., France;
⁹ trade name of Firmenich S.A., Switzerland;
¹⁰ trade name of PFW Aroma Chemicals B.V., the Netherlands.

More particularly, the advantages of the invention are manifested in the case of aroma chemicals that are selected from volatile fragrances and aroma mixtures comprising at least one volatile fragrance. Volatile fragrances are understood to mean fragrances having a high vapor pressure at room temperature. A fragrance is considered to be a volatile fragrance especially when it has the following property: If a droplet of the volatile fragrance is applied to a strip of paper and left to evaporate off under ambient conditions at room temperature (22° C.), its odor is no longer perceptible to an experienced perfumer 2 hours after application. The volatile fragrances especially include the following compounds: rose oxide (tetrahydro-4-methyl-2-(2-methylpropenyl)-2H-pyran), 4-methyl-2-(2-methylpropyl)oxane or 4-methyl-2-(2-methylpropyl)-2H-pyran (Dihydrorosan®), prenyl acetate (=3-methylbut-2-enyl acetate), isoamyl acetate, dihydromyrcenol (2,6-dimethyloct-7-en-2-ol) and methylheptenone (6-methylhept-5-en-2-one). If an aroma mixture comprising at least one volatile fragrance is used for loading, the proportion of the volatile fragrance is generally at least 1% by weight, especially at least 5% by weight, for example 1% to 99% by weight, especially 5% to 95% by weight, based on the total weight of the aroma chemical mixture used for loading.

Further odorants or aroma chemicals with which the odorants mentioned can be combined to give an odorant composition can be found, for example, in S. Arctander, Perfume and Flavor Chemicals, Vol. I and II, Montclair, N. J., 1969, Author's edition or K. Bauer, D. Garbe and H. Surburg, Common Fragrance and Flavor Materials, 4th. Ed., Wiley-VCH, Weinheim 2001. Specifically, the following may be mentioned:

Extracts from natural raw materials such as essential oils, concretes, absolutes, resins, resinoids, balsams, tinctures, for example ambra tincture; amyris oil; angelica seed oil; Angelica root oil; anise oil; valerian oil; basil oil; tree moss absolute; bay oil; mugwort oil; benzoin resin; bergamot oil; beeswax absolute; birch tar oil; bitter almond oil; savory oil; bucco leaf oil; cabreuva oil; cade oil; calamus oil; camphor oil; Cananga oil; cardamom oil; cascarilla oil; Cassia oil; cassie absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill oil; dill seed oil; eau de brouts absolute; oakmoss absolute; elemi oil; estragon oil; Eucalyptus citriodora oil; Eucalyptus oil; fennel oil; spruce needle oil; Galbanum oil; Galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; Camellia oil blue; Camellia oil roman; carrot seed oil; cascarilla oil; pine needle oil; spearmint oil; cumin oil; labdanum oil; labdanum absolute; labdanum resin; lavandin absolute; lavandin oil; lavender absolute; lavender oil; lemon grass oil; lovage oil; lime oil distilled; lime oil pressed; linalool oil; Litsea cubeba oil; laurel leaf oil; macis oil; marjoram oil; mandarin oil; Massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; oregano oil; palmarosa oil; patchouli oil; Perilla oil; Peruvian balsam oil; parsley leaf oil; parsley seed oil; petitgrain oil; peppermint oil; pepper oil; allspice oil; pine oil; poley oil; rose absolute; rosewood oil; rose oil; rosemary oil; sage oil dalmatian; sage oil Spanish; sandalwood oil; celery seed oil; spike lavender oil; star anise oil; Styrax oil; Tagetes oil; fir needle oil; tea tree oil; turpentine oil; thyme oil; tolu balsam; tonka absolute; tuberose absolute; vanilla extract; violet leaf absolute; Verbena oil; vetiver oil; juniper berry oil; wine yeast oil; vermouth oil; wintergreen oil; ylang oil; hyssop oil; civet absolute; cinnamon leaf oil; cinnamon bark oil; and fractions thereof or ingredients isolated therefrom.

Individual odorants are, for example, those from the group of
the hydrocarbons, for example 3-carene; alpha-pinene; beta-pinene; alpha-terpinene; gamma-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;
the aliphatic alcohols, for example hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
the aliphatic aldehydes and acetals thereof, for example hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trimethyl-4-hexene; citronellyloxyacetaldehyde; (E/Z)-1-(1-methoxypropoxy)-3-hexene; the aliphatic ketones and oximes thereof, for example 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
the aliphatic sulfur-containing compounds, for example 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
the aliphatic nitriles, for example 2-nonenenitrile; 2-undecenenitrile; 2-tridecenenitrile; 3,12-tridecadienenitrile; 3,7-dimethyl-2,6-octadienenitrile; 3,7-dimethyl-6-octenenitrile;
the esters of aliphatic carboxylic acids, for example (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E) and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; (E/Z)-ethyl 2,4-decadienoate; methyl 2-octynoate; methyl 2-nonynoate; allyl 2-isoamyloxyacetate; methyl 3,7-dimethyl-2,6-octadienoate; 4-methyl-2-pentyl crotonate;
the acyclic terpene alcohols, for example geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2-butenoates thereof;
the acyclic terpene aldehydes and ketones, for example geranial; neral; citronellal; 7-hydroxy-3,7-dimethyloctanal; 7-methoxy-3,7-dimethyloctanal; 2,6,10-trimethyl-9-undecenal; geranyl acetone; and also the dimethyl and diethyl acetals of geranial, neral, 7-hydroxy-3,7-dimethyloctanal; the cyclic terpene alcohols, for example menthol; isopulegol; alpha-terpineol; terpineol-4; menthan-8-ol; menthan-1-ol; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;
the cyclic terpene aldehydes and ketones, for example menthone; isomenthone; 8-mercaptomenthan-3-one; carvone; camphor; fenchone; alpha-ionone; beta-ionone; alpha-n-methylionone; beta-n-methylionone; alphaisomethylionone; beta-isomethylionone; alpha-irone; alpha-damascone; betadamascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalene-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methyl cedryl ketone);
the cyclic alcohols, for example 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
the cycloaliphatic alcohols, for example alpha-3,3-trimethylcyclohexylmethanol; 1-(4-isopropylcyclohexyl)ethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
the cyclic and cycloaliphatic ethers, for example cineol; cedryl methyl ether; cyclododecyl methyl ether; 1,1-dimethoxycyclododecane; 1,4-bis(ethoxymethyl)cyclohexane; (ethoxymethoxy)cyclododecane; alpha-cedrene epoxide; 3a,6,6,9a-tetramethyldodecahydronaphtho[2,1-b]furan; 3a-ethyl-6,6,9a-tri-methyldodecahydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
the cyclic and macrocyclic ketones, for example 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; cis-3-methylpent-2-en-1-ylcyclopent-2-en-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tertpentylcyclohexanone; cyclohexadec-5-en-1-one; 6,7-dihydro-1,1,2,3,3-pentamethyl-4(5H)-indanone; 8-cyclohexadecen-1-one; 7-cyclohexadecen-1-one; (7/8)-cyclohexadecen-1-one; 9-cycloheptadecen-1-one; cyclopentadecanone; cyclohexadecanone;
the cycloaliphatic aldehydes, for example 2,4-dimethyl-3-cyclohexenecarbaldehyde; 2-methyl-4-(2,2,6-trimethylcyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;
the cycloaliphatic ketones, for example 1-(3,3-dimethylcyclohexyl)-4-penten-1-one; 2,2-dimethyl-1-(2,4-dimethyl-3-cyclohexen-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl (2,4-dimethyl-3-cyclohexen-1-yl) ketone;
the esters of cyclic alcohols, for example 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or -6-indenyl acetate; 4,7-methano-3a,4,5,6,7,7ahexahydro-5- or -6-indenyl propionate; 4,7-methano-3a,4,5,6,7,7a-hexahydro-5- or -6-indenyl isobutyrate; 4,7-methanooctahydro-5- or -6-indenyl acetate;
the esters of cycloaliphatic alcohols, for example 1-cyclohexylethyl crotonate;
the esters of cycloaliphatic carboxylic acids, for example allyl 3-cyclohexylpropionate; allyl cyclohexyloxyacetate; cis- and trans-methyl dihydrojasmonate; cis- and trans-methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexenecarboxylate; ethyl 2-methyl-1,3-dioxolane-2-acetate;
the araliphatic alcohols, for example benzyl alcohol; 1-phenylethyl alcohol, 2-phenylethyl alcohol, 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
the esters of araliphatic alcohols and aliphatic carboxylic acids, for example benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;
the araliphatic ethers, for example 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;
the aromatic and araliphatic aldehydes, for example benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl) propanal; 2-methyl-3-(4-isobutylphenyl)propanal; 3-(4-tert-butylphenyl) propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxy-benzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl) propanal;
the aromatic and araliphatic ketones, for example acetophenone; 4-methylacetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl) ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;
the aromatic and araliphatic carboxylic acids and esters thereof, for example benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
the nitrogen-containing aromatic compounds, for example 2,4,6-trinitro-1,3-dimethyl-5-tert-butylbenzene; 3,5-dinitro-2,6-dimethyl-4-tert-butylacetophenone; cinnamonitrile; 3-methyl-5-phenyl-2-pentenonitrile; 3-methyl-5-phenylpentanonitrile; methyl anthranilate; methyl N-methylanthranilate; Schiff's bases of methyl anthranilate with 7-hydroxy-3,7-dimethyloctanal, 2-methyl-3-(4-tert-butylphenyl)propanal or 2,4-dimethyl-3-cyclohexenecarbaldehyde; 6-isopropylquinoline; 6-isobutylquinoline; 6-sec-butylquinoline; 2-(3-phenylpropyl)pyridine; indole; skatole; 2-methoxy-3-isopropylpyrazine; 2-isobutyl-3-methoxypyrazine;
the phenols, phenyl ethers and phenyl esters, for example estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;
the heterocyclic compounds, for example 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
the lactones, for example 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis- and trans-11-pentadecen-1,15-olide; cis- and trans-12-pentadecen-1,15-olide; 1,16-hexadecanolide; 9-hexadecen-1,16-olide;
10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

In addition, suitable aroma chemicals are macrocyclic carbaldehyde compounds as described in WO 2016/050836.

Particular preference is given to mixtures of L-menthol and/or DL-menthol, L-menthone, L-menthyl acetate, or L-isopulegol, which are highly sought-after as analogs or substitutes for what are referred to as synthetic dementholized oils (DMOs). The mixtures of these minty compositions are preferably used in the ratio of L-menthol or DL-menthol 20-40% by weight, L-menthone 20-40% and L-menthyl acetate 0-20%, or in the ratio of 20-40% by weight, L-menthone 20-40% and L-isopulegol 0-20%.

The aforementioned aromas and aroma mixtures can be used as such or in a solvent which in itself is not an aroma. Typical solvents for aromas are especially those having a boiling point at standard pressure above 150° C. and which do not dissolve the wall material, e.g. diols such as propanediol and dipropylene glycol, C₈-C₂₂ fatty acid C₁-C₁₀-alkyl esters such as isopropyl myristate, di-C₆-C₁₀-alkyl ethers, e.g. dicapryl ether (Cetiol® OE from BASF SE), di-C₁-C₁₀-alkyl esters of aliphatic, aromatic or cycloaliphatic di- or tricarboxylic acids, for example dialkyl phthalates such as dimethyl and diethyl phthalate and mixtures thereof, dialkyl hexahydrophthalates, e.g. dimethyl cyclohexane-1,2-dicarboxylate, diethyl cyclohexane-1,2-dicarboxylate and diisononyl 1,2-cyclohexanedicarboxylate, and dialkyl adipates, such as dibutyl adipate (e.g. Cetiol® B from BASF SE), C₈-C₂₂ fatty acid triglycerides, e.g. vegetable oils or cosmetic oils such as octanoyl/decanoyltriglyceride (e.g. the commercial product Myritol® 318 from BASF SE), dimethyl sulfoxide and white oils.

In a further group of embodiments, the organic active of low molecular weight is an active pharmaceutical ingredient, API for short. Active pharmaceutical ingredients are typically active therapeutic ingredients, active diagnostic ingredients and active prophylactic ingredients, and corresponding combinations of active ingredients. The active pharmaceutical ingredient(s) may be in an amorphous state, a crystalline state or a mixture thereof. The active pharmaceutical ingredient(s) may be labelled with a detectable label such as a fluorescent label, a radioactive label or an enzymatic or chromatographically detectable species, and be used as a mixture with this label for loading of the microparticles.

The API may have a water solubility in deionized water of more than 10 mg/mL at 25° C. It is also possible to use active pharmaceutical ingredients having low water solubility as actives, for example those having a water solubility in deionized water of less than 10 mg/mL at 25° C. In any case, the API should have a partition coefficient with respect to the water-immiscible liquid and the aqueous phase of at least 1.0, in particular at least 2.0.

Preferred active therapeutic, diagnostic and prophylactic ingredients are those APIs that are suitable for parenteral administration. Representative examples of suitable APIs are the following categories and examples of APIs and alternative forms of these APIs, such as alternative salt forms, free acid forms, free base forms and hydrates:
analgesics/antipyretics; antiasthmatic drugs; antibiotics; antidepressants; antidiabetic drugs; antiphlogistics/inflammation inhibitors; antihypertensives; inflammation inhibitors; antineoplastics; antianxiety drugs; immunosuppressants; antimigraine drugs; tranquilizers/hypnotics; antitanginal drugs; antipsychotic drugs; antimanic drugs; antiarrhythmics; antiarthritic drugs; antigout drugs; anticoagulants; thrombolytic drugs; antifibrinolytic drugs; hemorheological drugs; antiplatelet drugs/thrombocyte aggregation inhibitors; anticonvulsives; anti-Parkinson's drugs; antihistamines/antipruritics; drugs for calcium regulation; antibacterial drugs; antiviral drugs; antimicrobial drugs; antiinfectives; bronchodilators; corticosteroids; steroidal compounds and hormones; hypoglycemic drugs; hypolipedemic drugs; proteins; nucleic acids; drugs useful for the stimulation of erythropoiesis; antiulcer drugs/antireflux drugs; antinausea drugs/antimosis drugs; oil-soluble vitamins and other medicaments.

Suitable active pharmaceutical ingredients are mentioned, for example, in WO 2007/070852, especially on pages 15 to 19. In addition, suitable active ingredients and drugs are listed in Martindale: The Extra Pharmacopoeia, 30th edition, The Pharmaceutical Press, London 1993.

In a further group of embodiments, the organic active is an agrochemical compound, i. e. an organic compound for crop protection, which is also termed as an organic crop protecting agent. Agrochemicals for encapsulation within the microparticles are, for example, pesticides, especially selected from the group consisting of fungicides, insecticides, nematicides, herbicides, pheromons, but also safeners, and growth regulators which can be included as single compounds but also as mixtures of different agrochemical compounds, for example as mixtures of two or more herbicides, mixtures of two or more fungicides, mixtures of two or more insecticides, mixtures of insecticides and fungicides, mixtures of one or more herbicides with a safener, and mixtures of one or more fungicides with a safener.

Typically, the agrochemicals are liquid or solid at 20° C. and 1 bar and are normally nonvolatile. The vapor pressure is typically below 0.1 mbar at 20° C., especially below 0.01 mbar. Agrochemicals, which are particularly suitable for being included in the microcapsule of the invention are sparingly water-soluble or even insoluble in water and, especially at 25° C., have a water solubility in deionized water of not more than 5 g/L and especially not more than 2 g/L.

Agrochemicals are known to those skilled in the art, for example from The Pesticide Manual, 17th edition, The British Crop Protection Council, London, 2015. Suitable crop protecting agents are listed, especially, in WO 2018/019629 on pages 10 to 15.

Examples of suitable insecticides are compounds from the classes of the carbamates, organophosphates, organochlorine insecticides, phenylpyrazoles, pyrethroids, neonicotinoids, spinosins, avermectins, milbemycins, juvenile hormone analogs, alkyl halides, organotin compounds, nereistoxin analogs, benzoylureas, diacylhydrazines, METI acaricides, and unclassified insecticides such as chloropicrin, pymetrozine, flonicamid, clofentezin, hexythiazox, etoxazole, diafenthiuron, propargite, tetradifon, chlorofenapyr, DNOC, buprofezine, cyromazine, amitraz, hydramethylnon, acequinocyl, fluacrypyrim, rotenone, or the agriculturally acceptable salts and derivatives thereof.

Examples of suitable fungicides are compounds from the classes of the dinitroanilines, allylamines, anilinopyrimidines, antibiotic fungicides, aromatic hydrocarbons, benzenesulfonamides, benzimidazoles, benzisothiazoles, benzophenones, benzothiadiazoles, benzotriazines, benzyl carbamates, carbamates, carboxamides, carboxylic acid diamides, chloronitriles, cyanoacetamide oximes, cyanoimidazoles, cyclopropanecarboxamides, dicarboximides, dihydrodioxazines, dinitrophenyl crotonates, dithiocarbamates, dithiolanes, ethylphosphonates, ethylaminothiazolecarboxamides, guanidines, hydroxy(2-amino)pyrimidines, hydroxyanilides, imidazoles, imidazolinones, isobenzofuranones, methoxyacrylates, methoxycarbamates, morpholines, N-phenylcarbamates, oxazolidinediones, oximinoacetates, oximinoacetamides, peptidylpyrimidine nucleosides, phenylacetamides, phenylamides, phenylpyrroles, phenylureas, phosphonates, phosphorothioates, phthalamic acids, phthalimides, piperazines, piperidines, propionamides, pyridazinones, pyridines, pyridinylmethylbenzamides, pyrimidineamines, pyrimidines, pyrimidinone hydrazones, pyrroloquinolinones, quinazolinones, quinolines, quinones, sulfamides, sulfamoyltriazoles, thiazolecarboxamides, thiocarbamates, thiophanates, thiophenecarboxamides, toluamides, triphenyltin compounds, triazines, triazoles and the agriculturally acceptable salts and derivatives thereof.

Examples of suitable herbicides are compounds from the classes of the acetamides, amides, aryloxyphenoxypropionates, benzamides, benzofurans, benzoic acids, benzothiadiazinones, bipyridylium salts, carbamates, chloroacetamides, chlorocarboxylic acids, cyclohexanediones, dinitroanilines, dinitrophenols, diphenyl ethers, glycines, imidazolinones, isoxazoles, isoxazolidinones, nitriles, N-phenylphthalimides, oxadiazoles, oxazolidinediones, oxyacetamides, phenoxycarboxylic acids, phenyl carbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl (thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, ureas and the agriculturally acceptable salts and derivatives thereof.

In a specific subgroup of this group of embodiments, the crop protecting agent is a crop protecting agent which is liquid at 22° C. and 1 bar or a mixture of two or more crop protecting agents which is liquid at 22° C. and 1 bar. Examples of room temperature liquid active ingredients are dimethenamid, especially the enantiomer thereof dimethenamid-P, clomazone, metolachlor, especially the enantiomer thereof S-metolachlor, alachlor, cinmethylin.

In a further specific subgroup of this group of embodiments, the crop protecting agent is a crop protecting agent or a mixture of crop protecting agents with low water solubility and a melting point of not more than 110° C. or a mixture of such active ingredients. These include, for example, pyrachlostrobin (64° C.), prochloraz (47° C.), metrafenon (100° C.), alphacypermethrin (79° C.) and pendimethalin (58° C.).

In yet a further specific subgroup of this group of embodiments, the crop protecting agent is a pheromone or a mixture of pheromones, optionally in combination with one or more attractants.

Pheromones are well known chemical compounds used for controlling undesired insects. For example, Metcalf, R. L. Ullmann's Encyclopedia of Industrial Chemistry 2000, keyword “Insect Control”, lists in Chapter 15.1 (Sex pheromone attractants) and Chapter 15.2 (Aggregation pheromones) suitable examples, wherein the pheromones for Lipidoptera in Table 4 are highly suitable.

Examples of pheromones include volatile alkanols and alkenols having from 5 to 18 carbon atoms, volatile alkanals and alkenals having from 5 to 18 carbon atoms, alkanones having from 6 to 18 carbon atoms, 1,7-dioxaspirononan and 3- or 4-hydroxy-1,7-dioxaspiroundecan, benzyl alcohol, Z-(9)-tricosene (muscalure), heneicosene, diacetyl, alcanoic acids having from 5 to 16 carbon atoms such as caprylic acid, laurylic acid, α-pinen, methyleugenol, ethyldodecanoate, tert-butyl 4-(or 5-)chloro-2-ethylcyclohexane-carboxylate, mycrenone, cucurbitacin, trimedlure (commercially available as Capilure®), and (E,E)-8,10-dodecadien-1-ol (codlemone).

Further examples of known pheromones are: Z-5-Decenyl acetate, dodecanyl acetate, Z-7-dodecenyl acetate, E-7-dodecenyl acetate, Z-8-dodecenyl acetate, E-8-dodecenyl acetate, Z-9-dodecenyl acetate, E-9-dodecenyl acetate, E-10-dodecenyl acetate, 11-dodecenyl acetate, Z-9,11-dodecadienyl acetate, E-9,11-dodecadienyl acetate, Z-11-tridecenyl acetate, E-11-tridecenyl acetate, tetradecenyl acetate, E7-tetradecenyl acetate, Z-8-tetradecenyl acetate, E-8-tetradacenyl acetate, Z-9-tetradecenyl acetate, E-9-tetradecenyl acetate, Z-10-tetradecenyl acetate, E-10-tetradecenyl acetate, Z-11-tetradecenyl acetate, E-11-tetradecenyl acetate, Z-12-pentadecenyl acetate, E-12-pentadecenyl acetate, hexadecanyl acetate, Z-7-hexadecenyl acetate, Z-11-hexadecenyl acetate, E-11-hexadecenyl acetate, octadecanyl acetate, E,Z-7,9-dodecadienyl acetate, Z,E-7,9-dodecadienyl acetate, E,E-7,9-dodecadienyl acetate, Z,Z-7,9-dodecadienyl acetate, E,E-8,10-dodecadienyl acetate, E,Z-9,12-dodecadienyl acetate, E,Z-4,7-tri-decadienyl acetate, 4-methoxy-cinnamaldehyde, [beta]-ionone, estragol, eugenol, indole, 8-methyl-2-decyl propanoate, E,E-9,11-tetradecadienyl acetate, Z,Z-9,12-tetradecadienyl acetate, Z,Z-7,11-hexadecadienyl acetate, E,Z-7,11-hexadecadienyl acetate, Z,E-7,11-hexadecadienyl acetate, E,E-7,11-hexadecadienyl acetate, Z,E-3,13-octadecadienyl acetate, E,Z-3,13-octadecadienyl acetate, E,E-3,13-octadecadienyl acetate, hexanol, heptanol, octanol, decanol, Z-6-nonenol, E-6-nonenol, dodecanol, 11-dodecenol, Z-7-dodecenol, E-7-dodecenol, Z-8-dodecenol, E-8-dodecenol, E-9-dodecenol, Z-9-dodecenol, E-9,11-dodecadienol, Z-9,11-dodecadienol, Z,E-5,7-dodecadienol, E,E-5,7-dodecadienol, E,E-8,10-dodecadienol, E,Z-8,1 0-dodecadienol, Z,Z-8,10-dodecadienol, Z,E-8,10-dodecadienol, E,Z-7,9-dodecadienol, Z,Z-7,9-dodecadienol, E-5-tetradecenol, Z-8-tetradecenol, Z-9-tetradecenol, E-9-tetradecenol, Z-10-tetradecenol, Z-11-tetradecenol, E-11-tetradecenol, Z-11-hexadecenol, Z,E-9,11-tetradecadienol, Z,E-9,12-tetradecadienol, Z,Z-9,12-tetradecadienol, Z,Z-10,12-tetradecadienol, Z,Z-7,11-hexadecadienol, Z,E-7,11-hexadecadienol, (E)-14-methyl-8-hexadecen-1-ol, (Z)-14-methyl-8-hexadecen-1-ol, E,E-10,12-hexadecadienol, E,Z-10,12-hexadecadienol, dodecanal, Z-9-dodecenal, tetradecanal, Z-7-tetradecenal, Z-9-tetradecenal, Z-11-tetradecenal, E-11-tetradecenal, E-11,13-tetradecadienal, E,E-8,10-tetradecadienal, Z,E-9,11-tetradecadienal, Z,E-9,12-tetradecadienal, hexadecanal, Z-8-hexadecenal, Z-9-hexadecenal, Z-10-hexadecenal, E-10-hexadecenal, Z-11-hexadecenal, E-11-hexadecenal, Z-12-hexadecenal, Z-13-hexadecenal, (Z)-14-methyl-8-hexadecenal, (E)-14-methyl-8-hexadecenal, Z,Z-7,11-hexadecadienal, Z,E-7,11-hexadecadienal, Z,E-9,11-hexadecadienal, E,E-10,12-hexadecadienal, E,Z-10,12-hexadecadienal, Z,E-10,12-hexadecadienal, Z,Z-10,12-hexadecadienal, Z,Z-11,13-hexadecadienal, octadecanal, Z-11-octadecenal, E-13-octadecenal, Z-13-octadecenal, Z-5-decenyl-3-methyl butanoate disparlure: (+) cis-7,8-epoxy-2-methyloctadecane, seudenol: 3-methyl-2-cyclohexen-1-ol, sulcatol: 6-methyl-5-hepten-2-ol, ipsenol: 2-methyl-6-methylene-7-octen-4-ol, ipsdienol: 2-methyl-6-methylene-2,7-octadien-4-ol, grandlure I: cis-2-isopropenyl-1-methylcyclobutane-ethanol, grandlure II: Z-3,3-dimethyl-1-cyclohexane-ethanol, grandlure III: Z-3,3-dimethyl-1-cyclohexane-acetalde-hyde, grandlure IV: E-3,3-dimethyl-1-cyclohexaneacetaldehyde, cis-2-ver-benol: cis-4,6,6-trimethylbicyclo[3,1,1]hept-3-en-2-ol cucurbitacin, 2-methyl-3-buten-2-ol, 4-methyl-3-heptanol, cucurbitacin, 2-methyl-3-buten-2-ol, 4-methyl-3-heptanol, [alpha]-pinene: 2,6,6-trimethylbicyclo[3,1,1]hepten-2-ene, [alpha]-caryophyllene: 4,11,11-trimethyl-8-methylene-bicyclo[7,2,0]undecane, Z-9-tricosene, ([alpha]-multistriatin, 2-(2-endo,4-endo)-5-ethyl-2,4-dimethyl-6,8-dioxabicyclo[3,2,1]octane, methyleugenol: 1,2-dimethoxy-4-(2-propenyl)phenol, lineatin: 3,3,7-trimethyl-2,9-dioxatricyclo[3,3,1,0]nonane, chalcogran: 2-ethyl-1,6-dioxaspiro[4,4]nonane, frontalin: 1,5-dimethyl-6,8-dioxabicyclo[3,2,1]octane, endo-brevicomin: endo-7-ethyl-5-methyl-6,8-dioxabicyclo[3,2,1]octane, exo-brevicomin: exo-7-ethyl-5-methyl-6,8-dioxabicyclo[3,2,1]octane, (Z)-5-(1-decenyl)dihydro-2-(3H)-furanone, farnesol: 3,7,11-trimethyl-2,6,10-dodecatrien-1-ol, nerolidol 3,7-11-trimethyl-1,6,10-dodecatrien-3-ol, 3-methyl,6-(1-methylethenyl)-9-decen-1-ol acetate, (Z)-3-methyl-6-(1-methylethenyl)-3,9-decadien-1-ol acetate, (E)-3,9-methyl-6-(1-methyl-ethenyl)-5,8-decadien-1-ol acetate, 3-methylene-7-methyl-octen-1-ol propionate, (Z)-3,7-dimethyl-2,7-octadien-1-ol propionate and (Z)-3,9-dimethyl-6-(1-methyl-ethenyl)-3,9-decadlien-1-ol propionate.

Preferred pheromones are Z-9-dodecenyl acetate (commercially available as RAK® 1 from BASF SE), (E7,Z9)-dodecadienly acetate (commercially available as RAK® 2 from BASF SE), (E,E)-8,10-dodecadien-1-ol (commercially available as RAK® 3 from BASF SE) and Z-8-dodecenyl acetate.

Particularly preferred pheromone comprises (E,E)-8,10-dodecadien-1-ol, which is also known as codlemone or codlure, and commercially available (e.g. as CheckMate® CM-F from Suterra LLC, USA, lsomate®-C Plus from Pacific Biocontrol Corp. USA; RAK® 3 from BASF SE). Codlemone may be used in pure form, in technical quality or mixed with other pheromones.

The aforementioned pheromons may be combined with one or more attractants. Attractants are non-pesticidal materials which may act in one or several of the following ways: a) entice the insect to approach the composition or the material treated with the composition; b) entice the insect to touch the composition or the material treated with the composition; c) entice the insect to consume the composition or the material treated with the composition; and d) entice the insect to return to the composition or the material treated with the composition. Suitable attractants include non-food attractants and food attractants, also termed as feeding stimulants.

Suitable non-food attractants are usually volatile material. The volatile attractants act as a lure and their type will depend on the pest to be controlled in a known manner. Non-food attractants include for example flavors of natural or synthetic origin. Suitable flavors include meat flavor, yeast flavor, seafood flavor, milk flavor, butter flavor, cheese flavor, onion flavor, and fruit flavors such as flavors of apple, apricot, banana, blackberry, cherry, currant, gooseberry, grape, grapefruit, raspberry and strawberry.

Suitable food attractants include:
proteins, including animal proteins and plant proteins, e. g. in the form meat meal, fish meal, fish extracts, seafood, seafood extracts, or blood meal, insect parts, crickets powder, yeast extracts, egg yolk, protein hydrolysates, yeast autolysates, gluten hydrolysates, and the like;
carbohydrates and hydrogenated carbohydrates, in particular mono- and disaccharides such glucose, arabinose, fructose, mannose, sucrose, lactose, galactose, maltose, maltotriose, maltotetrose, maltopentose or mixtures thereof such as molasses, corn syrup, maple syrup, invert sugars, and honey; polysaccharides including starch such as potato starch, corn starch, and starch based materials such as cereal powders (e.g. wheat powder, maize powder, malt powder, rice powder, rice bran), pectines, and glycerol, hydrogenated mono- and oligosaccharides (sugar alcohols) such as xylitol, sorbitol, mannitol, isomaltolose, trehalose and maltitol as well as maltitol containing syrups;

Preferred attractants are ethyl 3-methylbutanoate, methyl salicylate, amyl acetate, limonene or fruit extracts (e.g. apple extracts made from dried and extracted apples, comprises fructose, glucose, sorbitol, and the flavor of apples). Mixtures of attractants are also suitable.

In a further group of embodiments, the organic active is an organic active suitable for cosmetic applications or an active mixture other than the aforementioned aromas. Preferred cosmetic actives for loading of the microparticles are especially active plant ingredients and plant extracts.

Examples of cosmetic actives are skin and hair pigmentation agents, tanning agents, bleaches, keratin-hardening substances, antimicrobial active ingredients, photofilter active ingredients, repellent active ingredients, hyperemic substances, keratolytic and keratoplastic substances, antidandruff active ingredients, antiphlogistics, keratinizing substances, antioxidative active ingredients and active ingredients acting as free-radical scavengers, skin moisturizing or humectant substances, regreasing active ingredients, deodorizing active ingredients, sebostatic active ingredients, plant extracts, antierythematous or antiallergic active ingredients and mixtures thereof.

Artificial tanning actives suitable for tanning the skin without natural or artificial irradiation with UV rays are, for example, dihydroxyacetone, alloxan and walnut shell extract. Suitable keratin-hardening substances are generally active ingredients as are also used in antiperspirants, for example potassium aluminum sulfate, aluminum hydroxychloride, aluminum lactate, etc. Antimicrobial active ingredients are used to destroy microorganisms and/or to inhibit their growth and thus serve both as preservatives and also as a deodorizing substance which reduces the formation or the intensity of body odor. These include, for example, customary preservatives known to the person skilled in the art, such as p-hydroxybenzoic esters, imidazolidinylurea, formaldehyde, sorbic acid, benzoic acid, salicylic acid, etc. Such deodorizing substances are, for example, zinc ricinoleate, triclosan, undecylenoic acid alkylolamides, triethyl citrate, chlorhexidine, etc. Suitable photofilter active ingredients are substances which absorb UV rays in the UV-B and/or UV-A region. Suitable UV filters are those mentioned above. Additionally suitable are p-aminobenzoic esters, cinnamic esters, benzophenones and camphor derivatives, and pigments which stop UV rays, such as titanium dioxide, talc and zinc oxide. Suitable repellent active ingredients are compounds capable of warding off or driving away certain animals, particularly insects, from humans. These include, for example, 2-ethyl-1,3-hexanediol, N,N-diethyl-m-toluamide, etc. Suitable hyperemic substances, which stimulate blood flow through the skin, are, for example, essential oils, such as dwarf pine, lavender, rosemary, juniperberry, roast chestnut extract, birch leaf extract, hayseed extract, ethyl acetate, camphor, menthol, peppermint oil, rosemary extract, Eucalyptus Oil, Etc. Suitable Keratolytic and Keratoplastic Substances are, for example, salicylic acid, calcium thioglycolate, thioglycolic acid and its salts, sulfur, etc. Suitable antidandruff active ingredients are, for example, sulfur, sulfur polyethylene glycol sorbitan monooleate, sulfur ricinol polyethoxylate, zinc pyrithione, aluminum pyrithione, etc. Suitable antiphlogistics, which counter skin irritations, are, for example, allantoin, bisabolol, Dragosantol, chamomile extract, panthenol, etc.

Further cosmetic actives are aspalatin, glycyrrhizin, caffeine, proanthocyanidin, hesperetin, rutin, luteolin, polyphenols, oleuropein, theobromine, bioflavonoids and polyphenols.

Examples of plant extracts are also acai extract (Euterpe oleracea), acerola extract (Malpighia glabra), field horsetail extract (Equisetum arvense), agarius extract (Agarius blazei murill), aloe extract (Aloe vera, Aloe Barbadensis), apple extract (Malus), artichoke leaf extract (Cynara scolymus), artichoke blossom extract (Cynara edulis), arnica extract (Arnica Montana), oyster extract (Ostrea edulis), baldrian root extract (Valeriana officinalis), bearberry leaf extract (Arctostaphylos uva-ursi), bamboo extract (Bambus vulgaris), bitter melon extract (Momordica charantia), bitter orange extract (Citrus aurantium), nettle leaf extract (Urtica dioica), nettle root extract (Urtica dioica), broccoli extract (Brassica oleracea), watercress extract (Rorippa nasturtium), painted nettle extract (Coleus forskohlii), capsicum extract (Capsicum frutescens), extract from Centella asiatica (Gotu Kola), cinchona extract, cranberry extract (Vaccinium vitis-daea), turmeric extract (Curcuma longa), damiana extract (Tunera diffusa), dragonfruit extract (Pitahaya), extract from Echinacea purpurea, wheat placenta extract, edelweiss extract (Leotopodium alpinum), ivy extract (Hedera helix), bindii extract (Tribulus terrestris), Garcinia cambogia extract (Garcinia cambogia), ginkgo extract (Ginkgo biloba), ginseng extract (Panax ginseng), pomegranate extract (Punica granatum), grapefruit extract (Citrus paradisi), griffonia extract (Griffonia simplicifolia), green tea extract (Camellia sinensis), guarana extract (Paullinia cupana), cucumber extract (Cucumis sativus), dog rose extract (Rosa canina), blueberry extract (Vaccinium myrtillus), hibiscus extract (Malvacea), mallow extract, honey extract, hops extract (Humulus), ginger extract (Zingiber officinale), Iceland moss extract (Cetraria islandica), jojoba extract (Simmondsia chinensis), St. John's Wort extract (Hypericum perforatum), coffee concentrate, cocoa bean extract (Theobroma cacao), cactus blossom extract, chamomile blossom extract (Matricaria recutita, Matricaria chamomila), carrot extract (Daucus carota), kiwi extract (Aperygidae), kudzu extract (Pueraria lobata), coconut milk extract, pumpkinseed extract (Curcurbita pepo), cornflower extract (Centaurea cyanus), lotus flower extract, dandelion extract (Taraxacum officinale), maca extract (Lepidium peruvianum), magnolia blossom extract, mango extracts, milk thistle extract (Silybum marianum), marigold extract (Calendula officiennalis), mate extract (Hex paraguariensis), butcher's broom extract (Rugcus aculeatus), sea algae extracts, cranberry extracts (Vaccinium macrocarpon), Moringa Oleifera extract, extract from Moschus Malve (Malva moschata), evening primrose oil extract (Azadirachta indica), nettle extract (Urticaceae), olive leaf extract (Olea europea), orange extract (hesperidin), orchid extract, papaya extract (Carica papaya), peppermint extracts, extract from Carica papaya (Geissospermum), bitter orange extract (Citrus aurantioum), lingonberry extract (Vaccinium vitas-ideea), African cherry extract (Prunus africana), sugar beet extracts, resveratrole extract (Polygonum cuspidatum), rooibos extract (Aspalasthus Linnearis), rose blossom extract, horse chestnut extract (Aesculus hippocastanum), rosemary extract (Rosemarinus Officinalis), red clover extract (Trifolium platense), red wine extract (Vitis vinifera), saw palmetto extract (Serenoa repens), lettuce extract (Lactuca sativa), sandalwood extract (Santalum rubrum), sage extract (Salvia officinalis), horsetail extract (Equisetum), yarrow extract (Achillea millefolium), black pepper extract (Piper nigrum), black tea extract, waterlily extract (Nymphaea), white willow bark extract (Salix Alba), liquorice extract (Glycyrrhiza), devil's claw extract (Harpagophytum procumbens), thyme extract (Thymus vulgaris), tomato extract (Lycopersicum esculentum), grapeseed extract (Vitis vinifera), grapeskin extract (Vitis vinifera), watercress (Rorippa amphibia), willow bark extract (Salix alba), wormwood extract (Artemisia absinthium), white tea extract, yam root extract (Dioscorea opposita), yohimbe extract (Pausinystalia yohimbe), witch hazel extract (Hamamelis), cinnamon extract (Cinnamomum cassia Presl), lemon extract (Citrus) and onion extract (Allium cepa).

In a further group of embodiments, the organic active of low molecular weight is a vitamin, in particular a lipophilic vitamin, such as vitamin A, vitamin D, vitamin E or vitamin K or a combination thereof.

In a further group of embodiments, the organic active of low molecular weight is an organic effect compound. Effect compounds are organic actives which do not belong to agrochemicals, aromachemicals, vitamins, AIPs and cosmetic actives. The groups of effect compounds are typically not admitted for use in agriculture, for administration to human beings, for cosmetic or dietary purposes. They include but are not limited to compounds for construction chemistry, in particular catalysts, but also dyes, UV stabilizers, polymerization inhibitors, oxidation stabilizers and the like. Preferred actives for being encapsulated into the microparticles for applications in construction chemistry are especially polymerization catalysts.

Useful polymerization catalysts include those suitable for curing of reactive resins, especially addition resins, condensation resins or oxidation-curing resins. For this purpose, the polymerization catalyst is a catalyst for a free-radical polymerization, a polycondensation and/or a polyaddition. The suitable catalysts for a free-radical polymerization especially include peroxide splitters, and the catalysts known from coatings technology for oxidatively drying oil and alkyd resins as driers or siccatives. Suitable polycondensation catalysts are catalysts for silicone condensation and crosslinking. Polyaddition catalysts used may, for example, be catalysts for curing of epoxy resins. In addition, polyaddition catalysts used may, for example, be urethanization catalysts customarily used in polyurethane chemistry. These are compounds that accelerate the reaction of the reactive hydrogen atoms of isocyanate-reactive components with the organic polyisocyanates.

Useful polymerization catalysts especially include tertiary amines, phosphines and organic metal salts.

Tertiary amines useful as polymerization catalysts, especially for polyadditions, are e. g. triethylamine, tributylamine, N,N-dimethylcyclohexylamine (DMCHA), N-methyldicyclohexylamine, N,N-dimethylbenzylamine (BDMA), N-methylmorpholine, Nethylmorpholine, N-cyclohexylmorpholine, 2,2′-dimorpholinodiethyl ether (DMDEE), N,N,N′,N′-tetramethylethylenediamine, N,N,N′,N′-tetramethylbutylenediamine, N,N,N′,N′-tetramethylhexylene-1,6-diamine, N,N,N′,N″,N″-pentamethyldiethylenetriamine (PMDETA), N,N,N′,N″,N″-pentamethyldipropylenetriamine (PMDPTA), N,N,N-tris(3-dimethylaminopropyl)amine, bis(2-dimethylaminoethyl) ether (BDMAEE), bis(dimethylaminopropyl)urea, 2,4,6-tris(dimethylaminomethyl)phenol, and its salt with 2-ethylhexanoic acid and isomers thereof, 1,4-dimethylpiperazine (DMP), N-methylimidazole, 1,2-dimethylimidazole, 1-methyl-4-(2-dimethylaminoethyl) piperazine, 1-azabicyclo[3.3.0]octane, 1,4-diazabicyclo[2.2.2]octane (DABCO), 1,8-diazabicyclo[5.4.0]undec-7-ene (DBU), 1,5-diazabicyclo[4.3.0]non-7-ene (DBN).

Further useful polymerization catalysts, especially for polyadditions, include: tris(dialkylamino)-s-hexahydrotriazines, especially 1,3,5-tris(3-[dimethylamino]propyl) hexahydrotriazine.

Useful phosphines as polymerization catalysts, especially for polyadditions, are preferably tertiary phosphines, such as triphenylphosphine or methyldiphenylphosphine.

Organic metal salts useful as polymerization catalysts preferably have the general formula

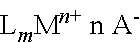

which
the ligand L is an organic radical or an organic compound selected from alkyl, alkenyl, alkynyl, aryl, arylalkyl, alkylaryl, heteroaryl, heteroarylalkyl, alkylheteroaryl and acyl, the ligand L having 1 to 20 carbon atoms, and the m ligands L being the same or different,
m is 0, 1, 2, 3, 4, 5 or 6,
M is a metal,
n is 1, 2, 3 or 4, and
the anion A⁻ is a carboxylate ion, alkoxylate ion or enolate ion.

The metal M is preferably selected from lithium, potassium, cesium, magnesium, calcium, strontium, barium, boron, aluminum, indium, tin, lead, bismuth, cerium, cobalt, iron, copper, lanthanum, manganese, mercury, scandium, titanium, zinc and zirconium; more particularly from lithium, potassium, cesium, tin, bismuth, titanium, zinc and zirconium.

The ligand L is preferably alkyl having 1 to 20 carbon atoms. More preferably L is alkyl having 1 to 10 carbon atoms, especially 1 to 4 carbon atoms, e.g. methyl, ethyl, n-propyl, isopropyl, n-butyl, isobutyl, sec-butyl or tert-butyl.

The carboxylate ion preferably has the formula R¹—COO⁻ where R¹ is selected from H, alkyl, alkenyl, alkynyl, aryl, arylalkyl, alkylaryl, heteroaryl, heteroarylalkyl, alkylheteroaryl and acyl, and where the R¹ radical has up to 20 carbon atoms, preferably 6 to 20 carbon atoms. Particularly preferred carboxylate ions are selected from the anions of natural and synthetic fatty acids, such as neodecanoate, isooctanoate and laurate, and the anions of resin acids and naphthenic acids.

The enolate ion preferably has the formula R²CH═CR³—O⁻ where R² and R³ are each selected from H, alkyl, alkenyl, alkynyl, aryl, arylalkyl, alkylaryl, heteroaryl, heteroarylalkyl, alkylheteroaryl and acyl, and where the R² and R³ radicals each have up to carbon atoms. Specific examples are ethylacetonate, heptylacetonate or phenylacetonate.

The enolate ion derives preferably from a 1,3-diketone having five to eight carbon atoms. Possible examples include acetylacetonate, the enolate of 2,4-hexanedione, the enolate of 3,5-heptanedione and the enolate of 3,5-octanedione.

The alkoxylate ion preferably has the formula R⁴—O— where R⁴ is selected from alkyl, alkenyl, alkynyl, aryl, arylalkyl, alkylaryl, heteroaryl, heteroarylalkyl, alkylheteroaryl and acyl, and where the R⁴ radical has up to 20 carbon atoms.

In particular embodiments the organic metal compound is selected from
alkali metal carboxylates, such as lithium ethylhexanoate, lithium neodecanoate, potassium acetate, potassium ethylhexanoate, cesium ethylhexanoate;
alkaline earth metal carboxylates, such as calcium ethylhexanoate, calcium naphthenate, calcium octoate (available as Octa-Soligen® Calcium from OMG Borchers), magnesium stearate, strontium ethylhexanoate, barium ethylhexanoate, barium naphthenate, barium neodecanoate;
aluminum compounds, such as aluminum acetylacetonate, aluminum dionate (e.g. K KAT® 5218 from King Industries);
zinc compounds, for example zinc(II) diacetate, zinc(II) ethylhexanoate and zinc(II) octoate, zinc neodecanoate, zinc acetylacetonate;
tin compounds, such as tin(II) carboxylates, examples being tin(II) acetate, tin(II) octoate, tin(II) ethylhexanoate, tin(II) neodecanoate, tin(II) isononanoate, tin(II) laurate, and dialkyltin(IV) salts of organic carboxylic acids, examples being dimethyltin diacetate, dibutyltin diacetate, dibutyltin dibutyrate, dibutyltin bis(2-ethylhexanoate), dibutyltin dilaurate, dibutyltin maleate, dioctyltin dilaurate and dioctyltin diacetate, especially dibutyltin dilaurate;
titanium compounds, such as tetra(2-ethylhexyl) titanate;
zirconium compounds, such as zirconium ethylhexanoate, zirconium neodecanoate, zirconium acetylacetonate (e.g. K-KAT® 4205 from King Industries);
zirconium dionates (e.g. K-KAT® XC-9213; XC-A 209 and XC-6212 from King Industries); zirconium 2,2,6,6-tetramethyl-3,5-heptanedionate;
bismuth compounds, such as bismuth carboxylates, especially bismuth octoate, bismuth ethylhexanoate, bismuth neodecanoate or bismuth pivalate (e.g. K-KAT® 348, XC-B221, XC-C₂₂₇, XC 8203, XK 651 from King Industries, TIB KAT 716, 716LA, 716XLA, 718, 720, 789 from TIB Chemicals, and those from Shepherd Lausanne);
manganese salts, such as manganese neodecanoate, manganese naphthenate;
cobalt salts, such as cobalt neodecanoate, cobalt ethylhexanoate, cobalt naphthenate;
iron salts, such as iron ethylhexanoate;
mercury compounds, such as phenylmercury carboxylate.

Preferred organic metal compounds are dibutyltin dilaurate, dioctyltin dilaurate, zinc(II) diacetate, zinc(II) dioctoate, zirconium acetylacetonate and zirconium 2,2,6,6-tetramethyl-3,5-heptanedionate, bismuth neodecanoate, bismuth dioctoate and bismuth ethylhexanoate.

In a first step i. of the process of the present invention invention, a water-immiscible liquid is prepared by mixing at least an organic active, the shell forming compound (SFC1) and, if necessary, one or more organic solvents. Preferably, the water-immiscible liquid is a solution of the organic active, the shell forming compound (SFC1) and the optional one or more organic solvents. For achieving the dissolution of the components, the mixture may be agitated and/or stirred.

Typically, the temperature for providing the water-immiscible liquid is not critical and typically in the range of 5 to 80° C.

The concentration of the organic active compound to be encapsulated in the water-immiscible liquid may vary and is typically in the range of 1 to 99% by weight, in particular in the range of 10 to 98.5% by weight, especially in the range of 20 to 98% by weight, based on the total weight of the water-immiscible liquid. The concentration of the the shell forming compound (SFC1) in the water-immiscible liquid is typically in the range of 1 to 50% by weight, in particular in the range of 1.5 to 25% by weight and especially in the range of 2 to 15% by weight, based on the total weight of the water-immiscible liquid. Preferably, the total amount of the shell forming compound (SFC1) and the organic active compound to be encapsulated is at least 10% by weight, in particular at least 20% by weight or at least 50% by weight, based on the total weight of the water-immiscible liquid. Typically the remainder, if necessary, is a water-immiscible organic solvent.

In a second step ii., the water-immiscible liquid obtained in step i. is emulsified in an aqueous medium. Thereby, an oil-in-water emulsion, hereinafter also termed o/w emulsion, of the water-immiscible liquid, which contains at least one organic active and the shell-forming compound (SFC1) is formed. In this o/w emulsion the water-immiscible liquid forms the disperse phase, while the aqueous phase is the continuous phase.

The aqueous phase usually contains at least one dispersant in order to stabilize the droplets of the o/w emulsion during its production in step ii., but also during the reaction of the shell forming compounds (SFC1) and (SFC2) in step iii.

Dispersants suitable for stabilizing o/w emulsions are common knowledge and are mentioned for example in EP 2794085 and EP 3007815, the teaching of which is expressly incorporated by reference. Typical dispersants include polysaccharides, polyvinyl alcohols, polymers bearing sulfonate groups, polymers bearing carboxylate groups, polyvinylpyrolidone, copolymers of vinylpyrrolidone and inorganic pickering stabilizers.

Suitable dispersants are typically water-soluble organic polymers. Inorganic pickering systems, such as colloidal silica and colloidal clay minerals may also be used as dispersants for this purpose. The pickering stabilizers, also referred to as pickering systems, may be used as such or in combination with the water-soluble organic polymers.

Dispersants of the group of polysaccharides include, for example, cellulose derivatives such as hydroxyethyl cellulose, methyl hydroxyethyl cellulose, methyl cellulose and carboxymethyl cellulose, methyl hydroxypropyl cellulose, lignin sulfonates and also mixtures of the above.

Preferred dispersants comprise at least one of partly or completely hydrolysed polyvinyl acetates (polyvinyl alcohols) and methyl hydroxy(C₁-C₄)alkyl celluloses as well as mixtures thereof. Preferred dispersants also comprise a pickering systems, in particular a combination of a pickering system with one or more of the aforementioned organic polymer dispersants, with preference given to partly or completely hydrolysed polyvinyl acetates (polyvinyl alcohols) and also methyl hydroxy(C₁-C₄)alkyl celluloses as well as mixtures thereof.

Amongst the organic water-soluble polymers, particular preference is given to partially hydrolysed polyvinyl acetates, also termed partially hydrolysed polyvinyl alcohols (PVAs) with particularly having a degree of hydrolysis of 70% to 99.9%, in particular 75 to 99%, more particular 80 to 95%. In addition, PVA copolymers, as described in WO 2015/165836, are also suitable. The PVA may be in particular a carboxy-modified anionic PVA. Such a carboxy-modified PVA preferably has a proportion of carboxyl groups of 1 to 6 mol %. In particular, a carboxy-modified PVA is used as a dispersant, whose 4% by weight aqueous solution preferably has a viscosity in the range of 20.0 to 30.0 mPa*s at 20° C. Amongst the group of partially hydrolysed polyvinyl alcohols particular preference is given to those having a degree of hydrolysis of 70% to 99.9%, in particular 75 to 99%, more particular 80 to 95% and especially 85 to 95%. An especially preferred dispersant is a carboxy modified anionic PVA having 1 to 6 mol-% of carboxyl groups, based on the amount of repeating units and a degree of hydrolysis of in the range of 75 to 99%, in particular 80 to 95% and especially 85% to 95%. Amongst these, those are preferred, whose 4% by weight aqueous solution has a viscosity of 20.0 to 30.0 mPa*s at 20° C.

Amongst the organic water-soluble polymers, particular preference is also given to methyl hydroxy(C₁-C₄)alkyl celluloses. Methyl hydroxy(C₁-C₄)alkyl celluloses are understood to mean methyl hydroxy(C₁-C₄)alkyl celluloses of a wide variety of degrees of methylation and also degrees of alkoxylation. The preferred methyl hydroxy(C₁-C₄)alkyl celluloses have an average degree of substitution DS of 1.1 to 2.5 and a molar degree of substitution MS of 0.03 to 0.9. Suitable methyl hydroxy(C₁-C₄)alkyl celluloses are for example methyl hydroxyethyl cellulose or methyl hydroxypropyl cellulose. A particularly preferred methyl hydroxypropyl cellulose is a methyl hydroxypropyl cellulose whose 2% aqueous solution has a viscosity in the range of 90 to 700 mPa·s, particularly in the range of 100 to 600 mPa·s, especially in the range of 400 to 550 mPa·s, as determined by Brookfield RVT at 20° C. at 20 rpm on bonedry basis. Particularly preferred is a methyl hydroxypropyl cellulose, whose 2% aqueous solution has a viscosity in the range of 400 to 550 mPa·s, as determined by Brookfield RVT at 20° C. at 20 rpm on bone-dry basis. This methyl hydroxypropyl cellulose is commercially available e.g. as Culminal MHPC 400 R of Ashland. Nonlimiting examples of commercially available methyl hydroxypropyl cellulose having a viscosity in the above-mentioned ranges are, for example, Culminal MHPC 100, Culminal MHPC 400 R, Culminal MHPC 500 RF of Ashland.

Also preferred dispersants are salts polymers bearing sulfonate groups. Such polymers include homo and copolymers of ethylenically unsaturated sulfonic acids, such as 2-acrylamide-2-methylpropane sulfonic acid, optionally with one or more water-soluble monomers, such as acrylamide or methacrylamide and the salts thereof. They also include the salts of lignin based sulfonic acids, also termed lignin sulfonates or lignosulfonates. Suitable lignin sulfonates may comprise, for example, sodium lignosulfonate, calcium lignosulfonate, ammonium lignosulfonate, magnesium lignosulfonate, potassium lignosulfonate, or sulfomethylated lignosulfonate. The aforementioned salts are in particular the sodium salts or the ammonium salts of the polymers bearing sulfonate groups.

Particularly suitable lignin based sulfonic acids have an average molar weight Mw of at least 5,000 Da. Preferably, an average molar weight Mw in the range of 5,000 Da to 100,000 Da, as determined by gel permeation chromatography according to DIN 55672-3. Preferably, said lignin based sulfonic acids have a degree of sulfonation from 1.0 to 2.5 mol per kilogram of said lignosulfonic acid, wherein the degree of sulfonation of the said lignin based sulfonic acid as applied herein is calculated from the sulfur content of said lignin based sulfonic acid as determined by atomic emission spectroscopy, from which the content of sulfate (determined according to DIN 38405-D5-2) is being subtracted. Preferred lignin based sulfonic acids are lignosulfonic acid, ethoxylated lignosulfonic acid or oxidized lignins. Particularly preferred lignin sulfonate is a sulfonated kraft lignin, which is commercially available e.g. as Reax® 910 of Ingevity. Non-limiting examples of commercially available lignin sulfonates include, for example, Greensperse s7, Reax® 85, Reax® 88A, Reax® 907, Reax® 910, Polyfon® o, Hyact, Kraftsperse 25m, and Borresperse NA. Greensperse s7, Kraftsperse 25m and Reax® 85 (commercially available from Ingevity), and Borresperse NA (commercially available from Borregaard AS) comprise sodium lignosulfonate. Reax® 88A, Reax® 907, Reax® 910, Polyfon® o and Hyact (commercially available from Ingevity) comprise sulfonated kraft lignin.

Amongst the organic water-soluble polymers, preference is also given to polymers bearing carboxyl groups. Typically, such polymers are homo- or copolymers of monoethylenically unsaturated carboxylic acids, such as acrylic acid, methacrylic acid, maleic acid, fumaric acid or itaconic acid. The polymers bearing carboxyl groups are typically used in their partially or completely neutralized form, where the carboxyl groups are converted in the anionic carboxylate form. Typically the counterions are selected from sodium and ammonium.

Another group of preferred dispersants are inorganic pickering systems, in particular a colloidal silica or a phyllosilikate.

In the context of this invention, the term “colloidal silica”, also termed colloidal silica dispersion, colloidal nano-particulate silica or a colloidal silica sol, refers to a stable dispersion of amorphous particulate silicon dioxide SiO₂ having particle sizes in the range 3 to 200 nm, preferably in the range of 5 to 170 nm, especially in the range of 10 to 150 nm. In this regard, the particle size of colloidal silica is at least 3 nm, preferably at least 5 nm and even more preferably at least 10 nm. The upper limit is set by the fact that the particles must be able to be present in a stable colloidal silica sol. Consequently, the particle size is at most 200 nm, preferably at most 170 nm and most preferably at most 150 nm, especially at most 100 nm. The particle size of the colloidal silica refers to the volume average particle diameter of the silica particles as determined by static light scattering, as described above.

An example of suitable colloidal silica sol is the colloidal silica sol having a particle size of 9 nm, which is commercially available e.g. under the trade name Bindzil® 30/360 from AkzoNobel. Another example of suitable colloidal silica sol is the colloidal silica sol having a particle size in the range of 10 to 150 nm, which is commercially available e.g. as Bindzil® 50/80 from AkzoNobel. Further suitable silica sols are Bindzil® 15/500, Bindzil® 30/220, Bindzil® 40/200, Bindzil® CC151HS, Bindzil® CC301 (AkzoNobel), Nyacol® 215, Nyacol® 830, Nyacol® 1430, Nyacol® 2034DI as well as Nyacol® DP5820, Nyacol® DP5480, Nyacol® DP5540 etc. (Nyacol Products), Levasil® 100/30, Levasil® 100 F./30, Levasil® 100S/30, Levasil® 200/30, Levasil® 200F/30, Levasil® 300F/30, Levasil® VP 4038, Levasil® VP 4055 (H.C. Starck/Bayer) or also CAB-O-SPERSE® PG 001, CAB-O-SPERSE® PG 002 (aqueous dispersions of CAB-O-SIL®, Cabot), Quartron PL-1, Quartron PL-3 (FusoChemical Co.), Köstrosol 0830, Köstrosol 1030, Köstrosol 1430 (Chemiewerk Bad Köstritz).

In a preferred embodiment, the dispersant comprises a colloidal silica sol having a particle size in the range of 3 to 200 nm, particularly in the range of 5 to 170 nm, especially in the range of 10 to 150 nm, which is commercially available e.g. as Bindzil 50/80 of AkzoNobel.

In another preferred embodiment the dispersant comprises a phyllosilicate, in particular a clay mineral which is capable of swelling in water, such as a hectorite, a montmorrilonite, a saponite or in particular a phyllosilicate having a high content of smectite, in particular a sodium smectite such as Laponite™ In particular, the inorganic pickering systems, such as the aforementioned colloidal silica and the phyllosilicates are used in combination with an organic polymer dispersant, in particular with an organic polymer dispersant from the group consisting of polyvinyl alcohols. Thereby, particularly dense microparticles are obtained. In this case, the particles of the inorganic pickering system remains at the surface of the microparticles.

In order to stabilize the o/w emulsion, the dispersant is added to the aqueous phase. The concentration of the dispersant in the aqueous phase is typically in the range of 0.1 to 10.0% by weight, in particular in the range of 0.2 to 5.0% by weight and especially in the range of 0.3 to 3.0% by weight, based on the total weight of the aqueous phase.

With regard to the amount of the water-immiscible liquid of step i. to be emulsified, the concentration of dispersant and the relative amount of the water-immiscible liquid of step i. to aqueous phase is preferably chosen such that the amount of the dispersant is in the range of 0.1 to 10% by weight, in particular in the range of 0.2 to 5% by weight, based on the total weight of the water-immiscible liquid of step i.

The weight ratio of the water-immiscible liquid prepared in step i. to the aqueous phase is typically in the range from 1:10 to 1.5:1, in particular in the range of 1:5 to 1.1:1, and especially in the range of 1:2 to 1:1.

To prepare the o/w emulsion in step ii. and for stabilization thereof one or more emulsifiers can be used together with the aforementioned dispersants. In contrast to dispersants, emulsifiers have typically a lower molecular weight of generally not more than 500 g/mol (number average). Preference is given to emulsifiers having an HLB value according to Griffin of at least 10, especially of at least 15. The HLB value (HLB=hydrophilic lipophilic balance) according to Griffin (W. C. Griffin: Classification of surface-active agents by HLB. In: J. Soc. Cosmet. Chem. 1, 1949, pp. 311-326) is a dimensionless number between 0 and 20 which provides information on the water and oil solubility of a compound. Preferably, these are non-ionic emulsifiers having an HLB value according to Griffin of at least 10, particularly of at least 15. However, also suitable are anionic and zwitterionic emulsifiers having an HLB value according to Griffin of at least 10, particularly of at least 15. Such emulsifiers are generally used in an amount from 0.1 to 10% by weight, especially 0.5 to 5% by weight, based on the total weight of the emulsion prepared in step ii. In general, the emulsifier or emulsifiers can be added to the water-immiscible liquid of step i before emulsifying or in the aqueous medium.

Examples of suitable emulsifiers having an HLB value according to Griffin of at least 10 are:
ethoxylated sorbitan fatty acid esters, particularly sorbitan mono-, di- and trifatty acid esters and mixtures thereof, such as sorbitan monostearate, sorbitan monooleate, sorbitan monolaurate, sorbitant tristearate, sorbitan sesquioleate, sorbitan dioleate, sorbitan trioleate;
lactyl esters of fatty acid monoesters of glycerol;
lecithins;
ethoxylated castor oils, ethoxylated hydrogenated castor oils with degrees of ethoxylation of at least 20, e. g. 20 to 60;
ethoxylated and/or propoxylated C₁₂-C₂₂-alkanols having degrees of alkoxylation in the range of at least 10, e.g. stearyl alcohol ethoxylate having a degree of ethoxylation in the range of 10 to 50, stearyl alcohol ethoxylate-copropoxylate having degrees of alkoxylation in the range of 10 to 50, isotridecyl ethoxylates having degrees of ethoxylation in the range of 10 to 50 and isotridecyl ethoxylate-co-propoxylates with degrees of alkoxylation in the range of 10 to 50;
ethoxylated and/or propoxylated C₄-C₁₆-alkylphenols having degrees of alkoxylation in the range of 10 to 50, e.g. nonylphenol ethoxylate having degrees of ethoxylation in the range of 10 to 50 and octylphenol ethoxylate having degrees of ethoxylation in the range of 10 to 50.

Typically, the aqueous phase is water, which preferably contains the dispersant and optionally the emulsifier. Additionally, the aqueous phase may contain a defoamer. The concentration of the defoamer in the aqueous phase is typically less than 2 g/kg, in particular not more than 1 g/kg, based on the total weight of the aqueous phase. The aqueous phase may contain small amounts of organic solvents, which are miscible with water. The amount of such solvents will generally not exceed 10% by weight, in particular not exceed 5% by weight or 1% by weight, based on the total weight of the aqueous phase.

The emulsification of the water-immiscible liquid provided in step i. in the aqueous medium to give the o/w emulsion in process step ii. can be effected by standard procedures for producing emulsion. Typically, emulsification is achieved by stirring or shearing a mixture of the aqueous phase and the solution obtained in step i. or combination of both.

The aqueous phase containing the dispersant is preferably initially charged into a vessel, and the water-immiscible liquid provided in step i. is metered into this aqueous phase with mixing of the two liquids, e.g. with stirring or shearing. It is also possible to continuously combine a stream of the aqueous phase and a stream of the solution of step i. in a mixing chamber and continuously remove the o/w-emulsion from the mixing chamber. The mixing chamber may have static or dynamic mixing elements.

Depending on the energy input into the mixture of the aqueous phase and the water-immiscible liquid provided in step i., it is possible to control the droplet size. Furthermore, the type and amount of dispersant described above influence the size of the emulsion droplets in equilibrium. A suitable amount can be chosen by routine. For the purpose of the invention, it has been found beneficial, if the final average particle size D[v, 0.5] of microparticles will not exceed 400 μm, in particular 200 μm and especially 100 μm.

Here and hereinafter, all figures for particle sizes, particle diameters and particle size distributions, including the D[v, 0.1], D[v, 0.5], D[v, 0.9], D[4,3] and D[3,2] values, are based on the particle size distributions ascertained by static laser light scattering to ISO 13320:2009 on samples of the microparticles. The abbreviation SLS is also used hereinafter for the expression “static laser light scattering to ISO 13320:2009”. In this connection, the D[v, 0.1] value means that 10% by volume of the particles of the measured sample have a particle diameter below the value reported as D[v, 01]. Accordingly, the D[v, 0.5] value means that 50% by volume of the particles of the measured sample have a particle diameter below the value reported as D[v, 0.5], and the D[v, 0.9] value means that 90% by volume of the particles of the measured sample have a particle diameter below the value reported as D[v, 0.9]. The D[4,3] value is the volume-weighted average determined by means of SLS, which is also referred to as the De Brouckere mean and corresponds to the mass average for the particles of the invention. The D[3,2] value is the surface-weighted average determined by means of SLS, which is also referred to as the Sauter mean diameter (SMD).

While stirring will usually produce droplets having an average droplet size D[v, 0.5] of at most or below 400 μm, in particular at most 200 μm and especially at most 100 μm, e.g. in the range of 0.5 to 400 μm, in particular 1 to 200 μm, especially 1 to 100 μm are achieved by means of apparatuses for generating a high shear field. It is also possible to introduce sufficient shear energy by vigorous stirring that average droplet sizes with D[v, 0.5] values in the range from 0.5 to 400 μm, preferably of 1 to 200 μm and especially 1 to 100 μm are achieved. Should even higher shear energy input be intended, it may be advantageous to use apparatuses for generating a high shear field.

By adjusting the droplet size of the o/w emulsion and droplet size distribution, it is possible to adjust the particle size and particle size distribution of the final microparticles containing the organic active compounds. In other words, a small average particle size of the microparticles is achieved by providing an o/w emulsion having a small average droplet size and likewise a narrow droplet size distribution of the o/w emulsion will result in a narrow particle size distribution of the obtained microparticles laden with the organic active compound.

Suitable stirrer types include propeller stirrers, impeller stirrers, disk stirrers, paddle stirrers, anchor stirrers, pitched-blade stirrers, cross-beam stirrers, helical stirrers, screw stirrers and others.

Suitable apparatuses for shearing, i.e. for generating a high shear field, are dispersing machines operating by the rotor-stator principle, i.e. rotor stator mixer, such as toothed ring dispersing machines also termed gear dispersing machines, further colloid mills and disk mills, high-pressure homogenizers, also termed high pressure mixers, and ultrasound homogenizers. High shear may also be achieved by using a dispersing disc or a cross-blade stirrer with one or multiple stages. High shear may also be achieved by passing the mixture through a microfluidic devices. Amongst apparatuses for shearing, preference is given to dispersing machines operating by the rotor-stator principle for generating the shear field, in particular to toothed ring dispersing machines. The diameter of the rotors and stators is typically in the range between 1 cm and 40 cm, depending on machine size and dispersing performance.

The speed of rotation of such dispersing machines is generally in the range from 500 to 20 000 rpm, in particular from 1000 to 15000 rpm (revolutions per minute), depending on the construction type. Of course, machines with large rotor diameters rotate at the lower end of the rotation speed range, while machines with small rotor diameters are usually operated at the upper end of the rotation speed range. The circumferential speed of the rotor is typically in the range of 5 to 50 m/s. The distance of the rotating parts from the stationary parts of the dispersing tool is generally 0.1 to 3 mm.

As mentioned above, droplet size can be controlled by the shear energy input into the mixture of the aqueous phase and the solution obtained in step i. The shear energy introduced can be directly derived from the power consumption of the apparatus for generating a shear field, taking account of the heat loss. Thus, the shear energy input into the o/w emulsion is preferably 250 to 25 000 watts h/m³ batch size. Particular preference is given to an energy input of 500 to 15 000, especially 800 to 10 000, watts h/m³ batch size, calculated based on the motor current.

In a preferred embodiment, the emulsification is carried out such that the emulsion droplets of the o/w emulsion have an average diameter D[v, 0.5], determined by means of light scattering, of at most or below 400 μm, e.g. in the range of 0.5 to 400 μm, in particular in the range of 1 to 200 μm and especially 1 to 100 μm. For this, the emulsification typically comprises mixing the solution of step i. with the aqueous phase and homogenization of the mixture. Homogenization is typically achieved by subjecting the mixture to high shear using a suitable device as described above. Mixing and homogenization can be carried out successively or simultaneously.

For example, the solution of step i. and the aqueous phase are mixed with stirring and the obtained emulsion is then homogenized as described herein, for example by
treatment of the emulsion with a rotor stator mixer, in particular a toothed-rim mixer;
applying ultrasound to the emulsion;
treatment of the emulsion with a dispersing disc or a cross-blade stirrer with one or multiple stages;
passing the emulsion through a membrane device,
passing the emulsion through a high-pressure homogenizer;
or by combinations thereof.

Mixing and homogenization can also be carried out simultaneously. For example, a stream of the aqueous phase and a stream of the solution of step i. are continuously combined in a mixing chamber, which is a homogenizer, and the o/w-emulsion is continuously removed from the mixing chamber. The above mentioned homogenizers can be used for this purpose and are adapted for continuous operation. For this purpose, principally any of the aforementioned homogenizers can be used. Preference is given to membrane devices and high pressure homogenizers.

The emulsification is usually carried out at a temperature in the range of 5 to 80° C., in particular in the range of 10 to 60° C. The temperature of the mixture is preferably selected such that it has a temperature in the range of 10 to 60° C., especially in the range of 10 to 40° C. Typically, the emulsification is carried out at atmospheric pressure or at a pressure above atmospheric pressure, e.g. at pressure up to 2 bar. However, a slight vacuum may be applied. Preferably, the vacuum will not be lower than 800 mbar.

In step iii. at least one second shell forming compound (SFC2) is added to the aqueous medium. The second shell forming compound (SFC2) may be added to the aqueous medium before or during carrying out step ii. Preferably, the second shell forming compound (SFC2) is added to the emulsion obtained in step ii., i. e. it is added to the emulsion after step ii. has been finished. Thereby, particularly uniform microparticles are obtained.

By the addition of the second shell forming compound (SFC2) to the o/w emulsion of step ii. it reacts with the first shell forming compound (SFC1) as explained above, whereby a shell is formed on the surface of the droplets of the water-immiscible liquid present in the emulsion obtained in step ii. of the process. Thereby, microparticles are obtained.

Typically the second shell forming compound (SFC2) may be added as such or it may also be added as an aqueous solution or emulsion depending on its solubility in water.

The second shell forming compound (SFC2) may be added all at once or within a certain time period, e. g. within 1 to 60 minutes, in particular within 5 to 30 minutes.

The temperature during the addition may vary depending on the reactivity of the shell forming compounds (SFC1) and (SFC2) and is typically in the range of 0 to 60° C., in particular in the range of 5 to 40° C. Higher temperatures will typically result in a more rapid reaction but potentially in a broader particle size distribution of the resulting microparticles. Particularly preferred, the temperature during addition will not exceed 30° C.

After the addition has been finished, the mixture of the o/w emulsion obtained in step ii. and the second shell forming compound (SFC2) may be allowed to react for a further period of time to complete the reaction of the shell forming compounds (SFC1) and (SFC2). This further period of time is also referred to as post reaction period. Typically, 24 h will be sufficient to achieve a complete reaction. During the post reaction period the temperature of the reaction mixture is typically in the range of 0 to 80° C., in particular in the range of 5 to 70° C.

During addition and during post reaction time, the mixture may be agitated or stirred to achieve a more uniform reaction and to avoid settling of the particles before the reaction of the shell forming compounds (SFC1) and (SFC2) is completed.

By the process of the invention an aqueous suspension of the microparticles is obtained. As explained above, the microparticles comprise a shell of an organic wall material which results from the reaction of the shell forming compounds (SFC1) and (SFC2) and a core containing the organic active compound.

The microparticles obtainable by the process of the invention preferably have a median particle diameter, i.e. a D[v, 05] value, in the range of 0.5 to 400 μm, particularly in the range of 0.8 to 200 μm, preferably in the range of 1.0 to 100 μm, more preferably in the range of 1.0 to 50 μm, especially in the range of 1.0 to 30 μm.

The microparticles obtainable by the process of the invention preferably have a Sauter mean diameter, i.e. a D[3,2] value, in the range of 0.3 to 300 μm, particularly in the range of 0.5 to 180 μm, more preferably in the range of 0.8 to 100 μm, more preferably in the range of 1.0 to 100 μm and especially in the range of 1.0 to 80 μm.

The microparticles obtainable by the process of the invention are preferably regular-shaped particles, especially sphere-shaped particles. The term “regular shaped” means that the surface of the particles does not have any great depressions in the wall material or elevations of wall material. The term “spherical” means that the particles have approximately the shape of a rotational ellipsoid and especially a spherical shape, where, in a particle in particular, the ratio of the longest axis through the centre of the particle to the shortest axis through the centre of the particle does not exceed a value of 2 and is especially in the range from 1:1 to 1.5:1.

As described above, step iii. results in an aqueous suspension of the microparticles. For further applications, the aqueous suspension may be used. However, it is also possible to isolate the microparticles from the aqueous suspension obtained in step iii. by conventional techniques. This further step may be carried out by the separation of the microparticles from the aqueous phase, e.g. by filtration or by centrifugation, or by evaporating the water of the aqueous suspension, e.g. in a spray-drying equipment. Regardless of which isolation technique is used, the microparticles may be dried. “Dried” is understood to mean that the laden microparticles comprise a residual amount of water of 5% by weight, preferably 1% by weight, based on the microparticles. The drying may for example be carried out in a stream of air and/or by applying a vacuum, optionally in each case with heating. This is accomplished, depending on the size of the microparticles, by means of convective driers such as spray driers, fluidized bed and cyclone driers, contact driers such as pan driers, paddle driers, contact belt driers, vacuum drying cabinet or radiative driers such as infrared rotary tube driers and microwave mixing driers. It may be also possible to remove residual water present after isolation of the laden polymer particles from the aqueous suspension by rinsing with ethanol or acetone, and/or blowing the microparticles dry with an inert gas such as air, nitrogen or argon. Optionally, for this purpose, pre-dried and/or pre-heated inert gases may also be used. The laden polymer microparticles may also be washed, preferably with aqueous propanediol solution, for example as a 10% by weight solution and optionally dried thereafter.

Surprisingly, the organoleptic profile is not significantly affected by the microencapsulation. In other words, the organoleptic profile of the laden microcapsules largely corresponds to that of the non-encapsulated aroma chemical and does not change significantly over time.

The present invention further provides compositions of the microparticles, obtainable by the process of the invention. The compositions of the invention preferably contain the active organic chemical in a total amount of 1 to 98% by weight, in particular 5 to 96% by weight, especially 10 to 90% by weight, based on the total weight of the microparticles. The constituents of the microparticles, i.e. the constituents of the composition other than solvents, are essentially the organic active, the wall material by the reaction of the shell forming compounds (SFC1) and (SFC2) and any auxiliaries that are used in the production of the microparticles and are not removed, such as the dispersants. The compositions of the invention may be in the form either of a suspension or of a powder, preference being given to suspension.

The present invention further provides products comprising a composition of the invention. Preference is given to products comprising the compositions of the invention in a proportion by weight of 0.01% to 80% by weight, based on the total weight of the product.

The nature of the product is naturally guided by the nature of the organic active. In case of aroma chemicals, it may be a product which typically comprises, for example, a perfume, a washing product, a cleaning product, a cosmetic product, a personal care product, a hygiene article, a food, a food supplement or a fragrance dispenser.

In case of agro chemicals, it is typically an agrochemical formulation containing at least one agrochemical in the form of microparticles obtainable by the process of the invention, optionally one or more further agrochemicals and suitable formulation auxiliaries. Suitable agrochemical formulations include capsule suspensions and solid formulations, such as powders or granules.

In case of pharmaceutical actives, it is typically a pharmaceutical formulation containing at least one pharmaceutical active in the form of microparticles obtainable by the process of the invention, optionally one or more further pharmaceutical actives and suitable formulation auxiliaries.

The present invention further provides for the use of compositions of the invention in the aforementioned products. Preference is given to the use of compositions of the invention in a product selected from perfumes, washing products, cleaning products, cosmetic products, personal care products, hygiene articles, foods, food supplements, fragrance dispensers and fragrances.

Compositions of the invention that comprise a fragrance as aroma chemical may be used in the production of perfumed articles. The olfactory properties and also the physical properties and the non-toxicity of the compositions of the invention underline their particular suitability for the end uses mentioned.

The use of the compositions is found to be particularly advantageous in conjunction with top notes of compositions, by way of example in perfume compositions comprising dihydrorosan, rose oxides or other readily volatile fragrances, e.g. isoamyl acetate, prenyl acetate or methylheptenone. In this case, the release of the important, sought-after top notes is effectively delayed. The fragrance or aroma compositions are accordingly metered in at the suitable point in the requisite amount.

In the mint compositions of L-menthol, DL-menthol, L-menthone and L-menthyl acetate described, aside from the aroma effect, a cooling effect is additionally applied in a targeted manner, e.g. in chewing gums, confectionery, cosmetic products, and industrial applications such as in textiles or superabsorbents. A further advantage lies in the high material compatibility of the compositions of the invention even with reactive or comparatively unstable components such as aldehydes, esters, pyrans/ethers, which may exhibit secondary reactions on the surfaces.

The positive properties contribute to use of the compositions of the invention with particular preference in perfume products, personal care products, hygiene articles, textile detergents and in cleaning products for solid surfaces.

The perfumed article is selected, for example, from perfume products, personal care products, hygiene articles, textile detergents and cleaning products for solid surfaces. Preferred perfumed articles of the invention are also selected from:
perfume products selected from perfume extracts, eau de parfums, eau de toilettes, eau de colognes, eau de solide, extrait parfum, air fresheners in liquid form, gel form or a form applied to a solid carrier, aerosol sprays, scented cleaners and scented oils;
personal care products selected from aftershaves, pre-shave products, splash colognes, solid and liquid soaps, shower gels, shampoos, shaving soaps, shaving foams, bath oils, cosmetic emulsions of the oil-in-water type, of the water-in-oil type and of the water-in-oil-in-water type, for example skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products, for example hairsprays, hair gels, setting hair lotions, hair conditioners, hair shampoo, permanent and semipermanent hair colorants, hair shaping compositions such as cold waves and hair smoothing compositions, hair tonics, hair creams and hair lotions, deodorants and antiperspirants, for example underarm sprays, roll-ons, deodorant sticks, deodorant creams, products of decorative cosmetics, for example eye shadows, nail varnishes, make-ups, lipsticks, mascara, toothpaste, dental floss;
hygiene articles selected from candles, lamp oils, joss sticks, propellants, rust removers, perfumed freshening wipes, armpit pads, baby diapers, sanitary towels, toilet paper, cosmetic wipes, pocket tissues, dishwasher deodorizer;
cleaning products for solid surfaces selected from perfumed acidic, alkaline and neutral cleaning products, for example floor cleaners, window cleaners, dishwashing detergents, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, limescale removers, grill and oven cleaners, algae and moss removers, mold removers, facade cleaners;
textile detergents selected from liquid detergents, powder detergents, laundry pretreatments such as bleaches, soaking agents and stain removers, fabric softeners, washing soaps, washing tablets.

In a further aspect, the compositions of the invention are suitable for use in surfactant-containing perfumed articles. This is because there is frequently a search—especially for the perfuming of surfactant-containing formulations, for example cleaning products (in particular dishwashing compositions and all-purpose cleaners)—for odorants and/or odorant compositions with a rose top note and marked naturalness.

In a further aspect, the compositions of the invention can be used as products for providing (a) hair or (b) textile fibres with an attractive odour note.

The compositions of the invention are therefore of particularly good suitability for use in surfactant-containing perfumed articles.

It is preferred if the perfumed article is one of the following:
an acidic, alkaline or neutral cleaning product selected in particular from the group consisting of all-purpose cleaners, floor cleaners, window cleaners, dishwashing detergents, bath and sanitary cleaners, scouring milk, solid and liquid toilet cleaners, powder and foam carpet cleaners, liquid detergents, powder detergents, laundry pretreatments such as bleaches, soaking agents and stain removers, fabric softeners, washing soaps, washing tablets, disinfectants, surface disinfectants,
an air freshener in liquid form, gel-like form or a form applied to a solid carrier or as an aerosol spray,
a wax or a polish selected in particular from the group consisting of furniture polishes, floor waxes and shoe creams, or
a personal care product selected in particular from the group consisting of shower gels and shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, aftersun creams and lotions, hand creams and lotions, foot creams and lotions, hair removal creams and lotions, aftershave creams and lotions, tanning creams and lotions, hair care products such as e.g. hairsprays, hair gels, setting hair lotions, hair conditioners, permanent and semipermanent hair colorants, hair shaping compositions such as cold waves and hair smoothing compositions, hair tonics, hair creams and hair lotions, deodorants and antiperspirants such as e.g. underarm sprays, rollons, deodorant sticks, deodorant creams, products of decorative cosmetics.

The customary ingredients with which odorants used in accordance with the invention, or odorant compositions of the invention, may be combined are common knowledge and are described for example in WO 2016/050836, the teaching of which is hereby expressly incorporated by reference.

Preference is likewise given to the use of compositions of the invention for controlled release of actives such as crop protecting agents and pharmaceutical agents.

### FIGURES

FIGS. 1a and 1b are microphotographies of the microparticles prepared by the protocol of example 2 at 2 different magnifications. The microphotography was taken by Scanning Electron Microscopy (SEM) as described below.

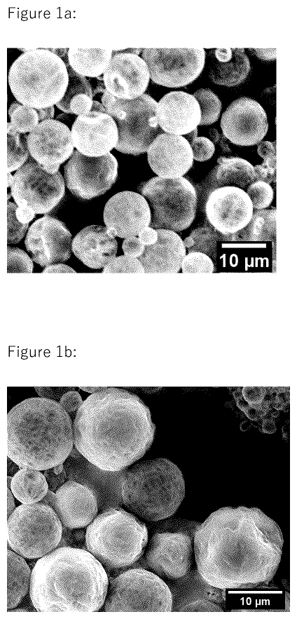

### EXAMPLES

### Materials

Cinmethylin: exo-(±)-1-Methyl-2-(2-methylbenzyloxy)-4-isopropyl-7-oxa-bicyclo[2.2.1]heptane;
Defoamer 1: Silicon SRE-PFL (Wacker Chemie AG);
Defoamer 2: 1-octanol
Hexyl salicylate: hexyl 2-hydroxybenzoate;
Laponite: synthetic phyllosilicate (LAPONITE-RD® BYK-Chemie GmbH);
Nile red: 9-(Diethylamino)-5H-benzo[a]phenoxazin-5-one used as lipophilic dye for fluorescence microscopy;
PVA 1: polyvinylalcohol, 88% hydrolyzed, Mw: 13,000 to 23,000;
PVA 2: polyvinylalcohol, 88% hydrolyzed, Mw: about 31,000 (Poval® 4-88, Kuraray);
PVP: Polyvinylpyrrolidone K 90 of BASF SE
Water: double distilled water, unless stated otherwise:

### Characterizations of the Prepared Microparticles

### Methods

### Microscopy and Particle Size Distribution

Scanning Electron Microscopy (SEM) was used to observe the morphology of the finished capsules. For this purpose an obtained microparticle slurry was diluted (1:20 w/w) with water and coated onto a glass slide using Polos spin 150i (2000 RPM for 20 s) spin-coater. The capsules were then sputter coated with a 5 nm thick platinum layer. The SEM images were recorded using a tabletop Hitachi TM3030 microscope.

Particle size distribution of the capsules is measured using a Malvern Mastersizer 2000 by laser diffraction according to ISO 13320 EN:2020-01. The data were treated according to the Mie-Theory by software using a “universal model” provided by Malvern Instruments. Important parameters are the dₙ-values for n=10, 50 and 90, the d₁₀, d₅₀ and d₉₀ values and the D[3,2] value, i. e. the Sauter Mean Diameter. The d₅e value is the volume-based average median d₅e particle size diameter which is also referred to as D[v,50].

Measuring the Release Rate of the Active from Microparticles

To determine the release rate the following release protocol was followed. 50 mg of the microparticle slurry obtained after the synthesis was added inside a cylindrical dialysis tube (diameter: 1 cm, length: 2 cm, Molecular weight cut off: 14 kDa) along with 2 mL of 36% (v/v) aqueous 1-propanol. This bag was dropped into 100 mL of 36% (v/v) 1-propanol and the whole solution was stirred mildly (~100 RPM) on a magnetic stirrer at room temperature. The outer media was continuously circulated through a calibrated UV-visible spectrophotometer (Cecil Instruments, Cambridge, UK) and the concentration of hexyl salicylate released into this medium from the capsules was recorded against time. For calculating the percent release of the oil from the capsules, 50 mg of the microparticle slurry was ultrasonicated in 100 mL 1-propanol for 1 hour and stirred overnight on an orbital shaker. Using the same spectrophotometer, the concentration of hexyl salicylate extracted into 1-propanol was recorded. This represented the maximum concentration of hexyl salicylate present in the slurry. Using this value as the denominator, percent hexyl salicylate released from the capsules was calculated.

As a control experiment, to realize the resistance offered by the microcapsule shell to the release, the same experiment was repeated by adding pure hexyl salicylate into the dialysis tube instead of the capsules and the rate of leakage of the unencapsulated oil was recorded for comparison.

### Microparticle Preparations

### Example 1

To prepare the water phase, 1.2 g of Laponite was dispersed in 140 g of water in a 250 mL beaker using an overhead stirrer (IKA Eurostar) equipped with a Rushton turbine. The dispersion was stirred at 1000 rpm for 20 min till a clear aqueous solution was obtained. Then, 2.59 g of 3.5 wt % aq. solution of sodium chloride, 7.5 g of 10 wt % aq. solution of PVA 1 and 2 drops of defoamer 2 were added, and the entire solution was placed in an ultrasonic bath for 5 min.

The oil phase was prepared by mixing 21.25 g of hexyl salicylate with 3.75 g of cyclohexyl isocyanate and 2 mg of Nile red dye.

The water phase was transferred to a jacketed reactor equipped with four baffles placed diametrically opposite to each other. Water was circulated externally in the reactor using a water bath (Julabo Me v.2) for temperature control. A temperature of 10° C. was maintained throughout. An oil-in-water emulsion was generated by adding the oil phase to the water phase in the reactor and homogenizing at 8000 RPM for 5 min using a silverson L4RT (rotor-stator dispersion tool). After emulsification the silverson was replaced by an overhead stirrer (IKA Eurostar) equipped with a Rushton turbine and the emulsion was stirred at 1000 RPM. Then, 2.16 g of 1,8-diaminooctane dissolved in 20 g of water was added into the emulsion using a syringe pump over 60 min. After addition the stirring speed was reduced to 600 RPM and maintained for 18 h.

Overall, 198 g of a uniform dispersion of spherical and monodisperse microcapsules (D[3,2]=1.5 μm) having an organic crystalline shell was obtained.

### Example 2

To prepare the water phase, 1.2 g of Laponite was dispersed in 140 g of water in a 250 mL beaker using an overhead stirrer (IKA Eurostar) equipped with a Rushton turbine. The dispersion was stirred at 1000 rpm for 20 min until a clear aqueous solution was obtained. Then, 2.59 g of 3.5 wt % aq. solution of sodium chloride, 7.5 g of 10 wt % aq. solution of PVA 1 and 2 drops of defoamer 2 were added, and the entire solution was placed in an ultrasonic bath for 5 min.

The oil phase was prepared by mixing 21.25 g of cinmethylin with 3.75 g of cyclohexyl isocyanate and 2 mg of Nile red dye.

The water phase was transferred to a jacketed reactor equipped with four baffles placed diametrically opposite to each other. Water was circulated externally in the reactor using a water bath (Julabo Me v.2) for temperature control. An oil-in-water emulsion was generated by adding the oil phase to the water phase in the reactor and homogenizing at 8000 RPM for 5 min using a Silverson L4RT (rotor-stator dispersion tool) with temperature maintained at 10° C. After emulsification the silverson was replaced by an overhead stirrer (IKA Eurostar) equipped with a Rushton turbine and the emulsion was stirred at 1000 RPM. Then, 2.16 g of 1,8-diaminooctane dissolved in 20 g of water was added into the emulsion using a syringe pump over 60 min. After addition the stirring speed was reduced to 600 RPM and temperature was increased to 20° C. These conditions were maintained for 18 h.

Overall, 198 g of a uniform dispersion of spherical and monodisperse microcapsules (D[3,2]=4 μm) having an organic crystalline shell was obtained.

D[v,10]=2.6 μm, D[v,50]=5.7 μm, D[v,90]=14.8 μm

### Example 3

To prepare the water phase, 1.2 g of Laponite was dispersed in 140 g of water in a 250 mL beaker using an overhead stirrer (IKA Eurostar) equipped with a Rushton turbine. The dispersion was stirred at 1000 rpm for 20 min until a clear aqueous solution was obtained. Then, 2.59 g of 3.5 wt % aq. solution of sodium chloride, 7.5 g of 10 wt % aq. solution of PVA1 and 2 drops of defoamer 2 were added, and the entire solution was placed in an ultrasonic bath for 5 min.

The oil phase was prepared by mixing 18.75 g of hexyl salicylate with 6.25 g of cyclohexyl isocyanate and 2 mg of Nile red dye.

The water phase was transferred to a jacketed reactor equipped with four baffles placed diametrically opposite to each other. Water was circulated externally in the reactor using a water bath (Julabo Me v.2) for temperature control. An oil-in-water emulsion was generated by adding the oil phase to the water phase in the reactor and homogenizing at 8000 RPM for 5 min using a Silverson L4RT (rotor-stator dispersion tool). A temperature of 10° C. was maintained during the emulsification. After emulsification the silverson was replaced by an overhead stirrer (IKA Eurostar) equipped with a Rushton turbine and the emulsion was stirred at 1000 RPM. Then, 3.61 g of 1,8-diaminooctane dissolved in 20 g of water was added into the emulsion using a syringe pump over 60 min at 10° C. After addition the stirring speed was reduced to 600 RPM and temperature was increased to 20° C. maintained for 18 h. Overall, 198 g of a uniform dispersion of spherical and monodisperse microcapsules (D[3,2]=2.3 μm) having an organic crystalline shell was obtained.

### Example 4

The water phase was prepared as follows. 142.5 g of water was introduced in a beaker. Under stirring using a silent crusher (rotor stator dispersion tool), 1.2 g of Laponite was added. The powder was dispersed for 10 min at 7.000 rpm. Then, 7.5 g of 10 wt % aq. solution of PVA 2 and 2 drops of defoamer 1 were added. The dispersion was placed in an ultrasonic bath (transonic 470/H) for 5 min.

The oil phase was obtained by mixing 21.25 g of dibutyl adipate with 3.75 g of cyclohexyl isocyanate.

The emulsion was generated by using a Silverson L5M-A (rotor stator dispersion tool). The oil phase was added to the water phase and homogenize for 7 min at 10,000 rpm.

Afterward the emulsion was transferred to a glass reactor equipped with an anchor stirrer. The media was cooled to 10° C. under stirring at 300 rpm. When the temperature was reached, a solution composed of 2,16 g of 1,8-diaminooctane and 20 g of water was added using a syringe pump over 60 minutes. The stirring and temperature were maintained during the addition and for an additional 4 hours. Afterwards, the bath was removed, the temperature slowly raised to room temperature 23° C. and the stirring was maintained for an additional 15 hours.

Overall, 198 g of a uniform dispersion of almost spherical and monodisperse microcapsules (D[3,2]=11.9 μm) having an organic crystalline shell were obtained.

### Example 5

The water phase was prepared as follows. 140 g of water was introduced in a beaker. Under stirring using a silent crusher (rotor stator dispersion tool), 1.2 g of Laponite was added. The powder was dispersed for 10 min at 7.000 rpm. Then, 2.59 g of 3.5 wt % aq. solution of sodium chloride, 7.5 g of 10 wt % aq. solution of PVA 2 and 2 drops of Defoamer 1 were added. The dispersion was placed in an ultrasonic bath (transonic 470/H) for 5 min.

The oil phase was obtained by mixing 21.25 g of dibutyl adipate with 3.75 g of cyclohexyl isocyanate.

The emulsion was generated by using a Silverson L5M-A (rotor stator dispersion tool). The oil phase was added to the water phase and homogenize for 7 min at 8,000 rpm.

Afterward the emulsion was transferred to a glass reactor equipped with an anchor stirrer. The media was cooled to 10° C. under stirring at 300 rpm. When the temperature was reached, a solution composed of 2.16 g of 1,8-diaminooctane and 20 g of water was added using a syringe pump over 60 minutes. The stirring and temperature were maintained during the addition and for an additional 4 hours. Afterwards, the bath was removed, the temperature slowly raised to room temperature 23° C. and the stirring was maintained for an additional 15 hours.

Overall, 198 g of a uniform dispersion of almost spherical and monodisperse microcapsules (D[3,2]=5.9 μm) having an organic crystalline shell were obtained.

### Example 6

To prepare the water phase, 4 g of 10 wt % aq. solution of PVA 1 was mixed with 80 g of water.

The oil phase was prepared by mixing 11.33 g of hexyl salicylate and 2 g of cyclohexyl isocyanate.

The water phase was transferred to a jacketed reactor. Water was circulated externally in the reactor using a water bath (Julabo Me v.2) for temperature control. An oil-in-water emulsion was generated by adding the oil phase to the water phase in the reactor and homogenizing at 5000 RPM for 5 min using a Silverson L4RT (rotor-stator dispersion tool). Temperature was maintained at 10° C. After emulsification the Silverson was replaced by an overhead stirrer (IKA Eurostar) equipped with a Rushton turbine and the emulsion was stirred at 180 RPM. Then 1.24 g 1,6-hexanediamine dissolved in 10 g water was added dropwise into the emulsion over 9 min. After addition the stirring and temperature were maintained for a further 18 h. Overall, 98 g of a uniform dispersion of spherical microcapsules (D[3,2]=9 μm) having an organic crystalline shell was obtained.

### Example 7

To prepare the water phase, 3 g of 10 wt % aq. solution of PVA 1 was mixed with 80 g of water.

The oil phase was prepared by mixing 8 g of hexyl salicylate and 2 g of cyclohexyl isocyanate.

The water phase was transferred to a jacketed reactor. Water was circulated externally in the reactor using a water bath (Julabo Me v.2) for temperature control. An oil-in-water emulsion was generated by adding the oil phase to the water phase in the reactor and homogenizing at 5000 RPM for 5 min using a Silverson L4RT (rotor-stator dispersion tool). Temperature was maintained at 12° C. After emulsification the Silverson was replaced by an overhead stirrer (IKA Eurostar) equipped with a Rushton turbine and the emulsion was stirred at 180 RPM. Then 1.38 g 1,8-diaminooctane dissolved in 10 g water was added dropwise into the emulsion over 15 min using a syringe pump. After addition the stirring and temperature were maintained for a further 18 h.

Overall, 100 g of a uniform dispersion of spherical microcapsules (D[3,2]=6 μm) having an organic crystalline shell was obtained.

### Example 8

To prepare the water phase, 3.6 g of 10 wt % aq. solution of PVA 1 was mixed with 80 g of water.

To prepare the oil phase 1.47 g of cyclohexyl isocyanate, 0.35 g of phenyl isocyanate and 10.3 g of hexyl salicylate were mixed together.

The water phase was transferred to a jacketed reactor. Water was circulated externally in the reactor using a water bath (Julabo Me v.2) for temperature control. An oil-in-water emulsion was generated by adding the oil phase to the water phase in the reactor and homogenizing at 5000 RPM for 5 min using a Silverson L4RT (rotor-stator dispersion tool). Temperature was maintained at 12° C. After emulsification the Silverson was replaced by an overhead stirrer (IKA Eurostar) equipped with a Rushton turbine and the emulsion was stirred at 180 RPM. Then 1.38 g 1,8-diaminooctane dissolved in 10 g water was added dropwise into the emulsion over 15 min using a syringe pump. After addition the stirring and temperature were maintained for a further 18 h.

Overall, 108 g of a uniform dispersion of spherical microcapsules (D[3,2]=5.8 μm) having an organic crystalline shell was obtained.

### Example 9

To prepare the water phase, 4 g of 10 wt % aq. solution of PVA1 was mixed with 80 g of water.

The oil phase was prepared by mixing 11.33 g of hexyl salicylate, 2 g of cyclohexyl isocyanate and 2 mg of Nile Red dye.

The water phase was transferred to a jacketed reactor. Water was circulated externally in the reactor using a water bath (Julabo Me v.2) for temperature control. An oil-in-water emulsion was generated by adding the oil phase to the water phase in the reactor and homogenizing at 5000 RPM for 5 min using a Silverson L4RT (rotor-stator dispersion tool). Temperature was maintained at 12° C. After emulsification the Silverson was replaced by an overhead stirrer (IKA Eurostar) equipped with a Rushton turbine and the emulsion was stirred at 180 RPM. Then 1.35 g 1,7-diaminoheptane dissolved in 10 g water was added dropwise into the emulsion over 15 min using a syringe pump. After addition the stirring and temperature were maintained for a further 18 h.

Overall, 108 g of a uniform dispersion of spherical microcapsules (D[3,2]=5.3 μm) having an organic crystalline shell was obtained.

### Example 10

To prepare the water phase, 4 g of 10 wt % aq. solution of PVA 1 was mixed with 80 g of water.

The oil phase was prepared by mixing 11.33 g of hexyl salicylate, 2 g of cyclohexyl isocyanate and 2 mg of Nile Red dye.

The water phase was transferred to a jacketed reactor. Water was circulated externally in the reactor using a water bath (Julabo Me v.2) for temperature control. An oil-in-water emulsion was generated by adding the oil phase to the water phase in the reactor and homogenizing at 5000 RPM for 5 min using a Silverson L4RT (rotor-stator dispersion tool). Temperature was maintained at 12° C. After emulsification the Silverson was replaced by an overhead stirrer (IKA Eurostar) equipped with a Rushton turbine and the emulsion was stirred at 180 RPM. Then 1.48 g 1,8-diaminooctane dissolved in 10 g water was added dropwise into the emulsion over 15 min using a syringe pump. After addition the stirring and temperature were maintained for a further 18 h.

Overall, 108 g of a uniform dispersion of spherical microcapsules (D[3,2]=5.2 μm) having an organic crystalline shell was obtained.

### Example 11

To prepare the water phase, 1.6 g of PVP was dissolved in 80 g of water.

The oil phase was prepared by mixing 11.33 g of hexyl salicylate and 2 g of cyclohexyl isocyanate.

The water phase was transferred to a jacketed reactor. Water was circulated externally in the reactor using a water bath (Julabo Me v.2) for temperature control. An oil-in-water emulsion was generated by adding the oil phase to the water phase in the reactor and homogenizing at 5000 RPM for 5 min using a Silverson L4RT (rotor-stator dispersion tool). Temperature was maintained at 20° C. After emulsification the silverson was replaced by an overhead stirrer (IKA Eurostar) equipped with a Rushton turbine and the emulsion was stirred at 180 RPM. Then 1.48 g 1,8-diaminooctane was dissolved in 10 g water was added dropwise into the emulsion over 5 min. After addition the stirring and temperature were maintained for a further 18 h.

Overall, 100 g of a uniform dispersion of spherical microcapsules (D[3,2]=4.3 μm) having an organic crystalline shell was obtained.

### Example 12

To prepare the water phase, 4 g of a 10 wt % aq. solution of PVA 1 was mixed with 80 g of water.

The oil phase was prepared by mixing 11.33 g of hexyl salicylate and 2 g of cyclohexyl isocyanate.

The water phase was transferred to a jacketed reactor. Water was circulated externally in the reactor using a water bath (Julabo Me v.2) for temperature control. An oil-in-water emulsion was generated by adding the oil phase to the water phase in the reactor and homogenizing at 5000 RPM for 5 min using a Silverson L4RT (rotor-stator dispersion tool). Temperature was maintained at 12° C. After emulsification the silverson was replaced by an overhead stirrer (IKA Eurostar) equipped with a Rushton turbine and the emulsion was stirred at 180 RPM. Then 0.92 g 1,8-diaminooctane and 0.16 g tris (2-amino-ethyl) amine were mixed together and dissolved in 10 g water. This amine solution was added dropwise into the emulsion over 15 min using a syringe pump. After addition the stirring and temperature were maintained for a further 18 h.

Overall, 105 g of a uniform dispersion of spherical microcapsules (D[3,2]=6 μm) having an organic crystalline shell was obtained.

### Example 13

To prepare the water phase, 0.64 g of Laponite was dispersed in 70 g of water in a 250 mL beaker using an overhead stirrer (IKA Eurostar) equipped with a Rushton turbine. The dispersion was stirred at 1000 rpm for 20 min until a clear aqueous solution was obtained. Then, 4.0 g of 10 wt % aq. solution of PVA1 were added, and the entire solution was placed in an ultrasonic bath for 5 min.

The oil phase was prepared by mixing 11.33 g of hexyl salicylate with 2.0 g of toluene-2,4-diisocyanate.

The water phase was transferred to a jacketed reactor. Water was circulated externally in the reactor using a water bath (Julabo Me v.2) for temperature control. An oil-in-water emulsion was generated by adding the oil phase to the water phase in the reactor and homogenizing at 5000 RPM for 5 min using a Silverson L4RT (rotorstator dispersion tool). A temperature of 12° C. was maintained during the emulsification. After emulsification the silverson was replaced by an overhead stirrer (IKA Eurostar) equipped with a Rushton turbine and the emulsion was stirred at 400 RPM. Then, 2.27 g of cyclohexylamine dissolved in 20 g of water was added into the emulsion using a syringe pump over 15 min at 12° C. After addition the temperature was increased to 30° C. maintained with stirring for 18 h.

Overall, 77 g of a uniform dispersion of spherical and monodisperse microcapsules (D[3,2]=3.1 μm) having an organic crystalline shell was obtained.

### Example 14

To prepare the water phase, 4 g of a 10 wt % aq. solution of PVA 1 was mixed with 80 g of water.

The oil phase was prepared by mixing 11.33 g of hexyl salicylate and 2 g of isophorone diisocyanate.

The water phase was transferred to a jacketed reactor. Water was circulated externally in the reactor using a water bath (Julabo Me v.2) for temperature control. An oil-in-water emulsion was generated by adding the oil phase to the water phase in the reactor and homogenizing at 5000 RPM for 5 min using a Silverson L4RT (rotor-stator dispersion tool). Temperature was maintained at 12° C. After emulsification the silverson was replaced by an overhead stirrer (IKA Eurostar) equipped with a Rushton turbine and the emulsion was stirred at 400 RPM. Then 2.27 g of cyclhexylamine was dissolved in 10 g water. This amine solution was added dropwise into the emulsion over 15 min using a syringe pump. After the addition was complete the temperature was raised to 20° C. the stirring and temperature were maintained for a further 18 h.

Overall, 95.2 g of a uniform dispersion of spherical microcapsules (D[3,2]=1.7 μm) having an organic crystalline shell was obtained.

### Example 15

To prepare the water phase, 4 g of a 10 wt % aq. solution of PVA 1 was mixed with 80 g of water.

The oil phase was prepared by mixing 11.33 g of hexyl salicylate and 2 g of sebacoyl dichloride.

The water phase was transferred to a jacketed reactor. Water was circulated externally in the reactor using a water bath (Julabo Me v.2) for temperature control. An oil-in-water emulsion was generated by adding the oil phase to the water phase in the reactor and homogenizing at 5000 RPM for 5 min using a Silverson L4RT (rotor-stator dispersion tool). Temperature was maintained at 12° C. After emulsification the silverson was replaced by an overhead stirrer (IKA Eurostar) equipped with a Rushton turbine and the emulsion was stirred at 400 RPM. Then 1.65 g of cyclhexylamine was dissolved in 10 g water. This amine solution was added dropwise into the emulsion over 15 min using a syringe pump. After the addition was complete the temperature was raised to 20° C. the stirring and temperature were maintained for a further 18 h.

Overall, 96 g of a uniform dispersion of spherical microcapsules (D[3,2]=9.6 μm) having an organic crystalline shell was obtained.

### Example 16

To prepare the water phase, 4 g of a 10 wt % aq. solution of PVA 1 was mixed with 80 g of water.

The oil phase was prepared by mixing 11.33 g of hexyl salicylate and 2 g of cyclohexyl isocyanate and 0.046 g of 1,4-diazabicyclo[2.2.2]octane (DABCO).

The water phase was transferred to a jacketed reactor. Water was circulated externally in the reactor using a water bath (Julabo Me v.2) for temperature control. An oil-in-water emulsion was generated by adding the oil phase to the water phase in the reactor and homogenizing at 5000 RPM for 5 min using a Silverson L4RT (rotor-stator dispersion tool). Temperature was maintained at 12° C. After emulsification the silverson was replaced by an overhead stirrer (IKA Eurostar) equipped with a Rushton turbine and the emulsion was stirred at 400 RPM. Then 1.18 g of 1.6 hexanediol was dissolved in 10 g water. This solution was added dropwise into the emulsion over 15 min using a syringe pump. After the addition was complete the temperature was raised to 20° C. the stirring and temperature were maintained for a further 18 h and thereafter raised to 50° C. and kept for 2 h.

Overall, 87 g of a uniform dispersion of spherical microcapsules (D[3,2]=3.8 μm) having an organic crystalline shell was obtained.

### Release Rates

The slurries obtained in Examples 1 (with laponite) and 10 (without laponite) were examined using the method for determining the release rate described above. To realize the resistance offered by the shells of the microparticles against release, the dissolution and leakage of unencapsulated hexyl salicylate was also observed in comparison using the same method. It was found that the microparticles of Examples 1 and 10, which were prepared with PVA 1 or a combination of PVA 1 and Laponite 1 gave almost similar results, since in both cases only less than 20% of hexyl salicylate had been released after about 5 hours, whereas in this point in time already about 90% of the unencapsulated hexyl salicylate had leaked out.

1. A process for producing microparticles comprising at least one organic active compound encapsulated by a shell of an organic wall material, which comprises
i. providing a water-immiscible liquid comprising the organic active compound to be encapsulated and at least one first shell forming compound (SFC1);
ii. emulsifying the water-immiscible liquid obtained in step i. in an aqueous medium to obtain an oil-in-water emulsion of the water-immiscible liquid in the aqueous medium; and
iii. adding at least one second shell forming compound (SFC2) to the aqueous medium before or during carrying out step ii. or to the emulsion obtained in step ii., whereby an aqueous suspension of the microparticles is obtained;
wherein either the first shell forming compound (SFC1) or the second shell forming compound (SFC2) has 1, 2, 3, 4, 5 or 6 first reactive groups (RG1) per molecule, which are selected from isocyanate groups, isothiocyanate groups, carbonyl halide groups and/_o carboxylic anhydride groups, while the other shell forming compound has 1, 2, 3, 4, 5 or 6 second reactive groups (RG2) per molecule which are selected from hydroxyl groups, thiol groups and/or primary amino groups, or
wherein either the first shell forming compound (SFC1) or the second shell forming compound (SFC2) has only one reactive group while the other shell forming compound has 1, 2, 3, 4, 5 or 6 reactive groups.

2. The process of claim 1, wherein the first shell forming compound (SFC1) has 1, 2, 3, 4, 5 or 6 first reactive groups (RG1) per molecule, while the second shell forming compound (SFC2) has 1, 2 3, 4, 5 or 6 second reactive groups (RG2) per molecule, provided that either the first shell forming compound (SFC1) or the second shell forming compound (SFC2) has only one reactive group while the other shell forming compound has 1, 2, 3, 4, 5 or 6 reactive groups.

3. The process of claim 1, wherein
i. the first reactive groups (RG1) are isocyanate groups and the second reactive groups (RG2) are selected from hydroxyl groups, thiol groups and/or primary amino groups, optionally where RG2 are an amino groups or a combination of amino groups and hydroxyl groups; or
ii. the first reactive groups (RG1) are selected from carbonyl halide groups and/or carboxylic anhydride groups and the second reactive groups (RG2) are primary amino groups.

4. The process of claim 3, wherein the first shell forming compound (SFC1) is selected from the group consisting of aliphatic monoisocyanates, cycloaliphatic monoisocyanates and aromatic monoisocyanates and where the second shell forming compound (SFC2) is selected from the group consisting of aliphatic diamines, aliphatic triamines, aliphatic tetramines, aliphatic pentamines, aliphatic hexamines, cycloaliphatic diamines, cycloaliphatic triamines, cycloaliphatic tetramines, cycloaliphatic pentamines, cycloaliphatic hexamines, aromatic diamines, aromatic triamines, aromatic tetramines, aromatic pentamines, and aromatic hexamines.

5. The process of claim 4, wherein the second shell forming compound (SFC2) is an aliphatic diamine or a combination of at least an aliphatic diamine and an aliphatic, cycloaliphatic or aromatic triamine.

6. The process of claim 1, which has at least one of the following features (a) to (d):
(a) the organic active compound to be encapsulated is selected from agrochemicals, aromachemicals, pharmaceutically active compounds, vitamins, cosmetic actives and or organic effect compounds
(b) the water-immiscible liquid is a solution of the organic compound to be encapsulated and the first shell forming compound;
(c) the organic active compound to be encapsulated is a water-immiscible liquid at 25° C. and 1 bar; and or
(d) the weight ratio of the shell forming compounds to the water-immiscible liquid is in the range of 1:1 to 1:50.

7. The process of claim 6, wherein the organic compound to be encapsulated is an aroma chemical or an agrochemical.

8. The process of claim 1, wherein the emulsification step ii. is carried out in the presence of at least one dispersant, optionally selected from the group consisting of polysaccharides, polyvinyl alcohols, polymers bearing sulfonate groups, polymers bearing carboxylate groups, polyvinylpyrolidone copolymers of vinylpyrrolidone and inorganic pickering stabilizers.

9. The process of claim 8, wherein the dispersant comprises at least a phyllosilicate.

10. The process of claim 1, wherein emulsification comprises mixing the solution of step i. with the aqueous phase and homogenization of the mixture.

11. Microparticles comprising at least one organic active compound encapsulated by a shell of an organic wall material formed by the reaction of at least one first shell forming compound (SFC1) with at least one second shell forming compound (SFC2), where either the first shell forming compound (SFC1) or the second shell forming compound (SFC2) has 1 to 6 first reactive groups (RG1) per molecule, which are selected from isocyanate groups, isothiocyanate groups, carbonyl halide groups and/or carboxylic anhydride groups, while the other shell forming compound has 1 to 6 second reactive groups (RG2) per molecule which are selected from primary amino groups and/_or thiol groups,
wherein either the first shell forming compound (SFC1) or the second shell forming compound (SFC2) has only one reactive group while the other shell forming compound has 1, 2, 3, 4, 5 or 6 reactive groups.

12. The microparticles of claim 11, wherein first reactive groups (RG1) are isocyanate groups or isothiocyanate groups and the second reactive groups (RG2) are primary amino groups.

13. The microparticles of claim 11, which have an average particle size D[3,2](Sauter Mean Diameter) in the range of 0.3 to 300 μm.

14. The microparticles of claim 11, wherein the encapsulated organic compound is selected from agrochemicals, aromachemicals, pharmaceutically active compounds, vitamins, cosmetic actives and/or organic effect compounds.

15. The microparticles of claim 11, which comprise particles of an inorganic pickering system.

16. A product comprising microparticles as defined in claim 11 in a proportion of 0.1% to 80% by weight based on the total weight of the product.

17. A method comprising using the microparticles according to claim 11 as an additive for imparting a scent or a flavour to a product selected from perfumes, washing and cleaning products, cosmetic products, personal care products, hygiene articles, foods, food supplements, fragrance dispensers and/or fragrances, wherein the microparticles comprise an encapsulated aroma chemical.

18. A method for controlled release of an organic active compound comprising using the microparticles according to claim 11.
